(19) United States
(12) Patent Application Publication
(10) Pub. No.: US 2025/0235516 A1
(43) Pub. Date: Jul. 24, 2025

(54) PROTEIN-BASED ADVANCED WOUND HEALING SYSTEM

(71) Applicant: University of South Florida, Tampa, FL (US)

(72) Inventors: Graham Leonard Strauss, Tampa, FL (US); Piyush Koria, Tampa, FL (US)

(21) Appl. No.: 19/061,292

(22) Filed: Feb. 24, 2025

Related U.S. Application Data
(63) Continuation of application No. PCT/US2023/072606, filed on Aug. 22, 2023.
(60) Provisional application No. 63/399,850, filed on Aug. 22, 2022.

(51) Int. Cl. A61K 38/57; A61K 35/28; A61P 17/02; C07K 14/47; C07K 14/78; C07K 14/81

(52) U.S. Cl. CPC A61K 38/57; A61K 35/28; A61P 17/02; C07K 14/4711; C07K 14/78; C07K 14/8107; C07K 14/8146; C07K 2319/00

(57) ABSTRACT

A novel composition and method of enhancing wound healing and minimizing rejection of an implant is presented. The composition is a dry acellular mixture comprised of conditioned media from mesenchymal stem cells, a multifunctional protease inhibitor, and a tethering peptide that acts as a tether to keep the composition in contact with the targeted area. An antimicrobial fusion peptide may also be added to the composition.

### CROSS REFERENCE TO RELATED APPLICATIONS

This application is a continuation of and claims priority to International Patent Application No. PCT/US2023/072606, entitled “Protein-Based Advanced Wound Healing System”, filed Aug. 22, 2023, which is a nonprovisional of and claims priority to U.S. Provisional Patent Application Ser. No. 63/399,850, entitled “Protein-Based Advanced Wound Healing System”, filed Aug. 22, 2022, the contents of which are hereby incorporated by reference into this disclosure.

### GOVERNMENT SUPPORT

This invention was made with Government support under Grant No. R21AR068013-01A1 awarded by the National Institutes of Health (NIH). The Government has certain rights in the invention.

### SEQUENCE LISTING

The sequence listing entitled “Protein-Based Advanced Wound Healing System” in XML format, created on Aug. 18, 2023, and being 8000 bytes in size, is hereby incorporated by reference into this disclosure.

### FIELD OF INVENTION

This invention relates to a composition and method for wound healing. Specifically, the invention provides a novel composition, system and methods of use thereof in wound healing.

### BACKGROUND OF THE INVENTION

Diseases that are complex in cause often require drug combinations for treatment.¹ Drug combinations provide the term “broad spectrum treatment” in a conventional response to complex causes of disease. Combination therapies are considered a mixture of biologics, drugs, or both by combining them heterogeneously to broaden the therapeutic effect.² For example, a persistence in the body's inability to replace damaged tissue in a timely manner result in chronic wounds. This may be caused by other underlying diseases such as autoimmune disorders, poor vascularization, or imbalances in metabolic processes as seen in diabetes.³ All the latter may require systemically delivered medications to treat these underlying diseases, so it is advantageous to treat resulting chronic wounds with a point specific and broad spectrum therapeutic in order to decrease interactions between therapeutics in other areas of the body.

Moreover, chronic wounds also present high activity of protein degrading enzymes known as proteases. Proteases are an important part of tissue remodeling and repair as they are responsible for breaking down the extracellular matrix (ECM) making it possible for tissue remodeling. Wound healing is an intricate set of processes with diverse amounts of proteases and protease inhibitors that are involved in proper wound healing processes such as the need for proteases to tear down old or damaged tissue,⁴ but with limitation and means of inhibition^{8, 9}. Therefore, there exists a fine balance between proteases and protease inhibitors to accomplish the successful breaking down and remodeling of the ECM, making protease levels an important factor when evaluating the cause of chronicity of a wound.³⁸ Therefore, if the rate of ECM destruction is greater than the rate of remodeling, then a wound will not heal, which leads to chronicity^{8,9} and the absence of a well-developed ECM.⁹ Several studies have shown a correlation between high levels of proteases and nonhealing wounds.^{6,8-12} High levels of neutrophil derived proteases, neutrophil elastase,¹³ and matrix-metalloprotease (MMP) 11 have been associated with increased probability of nonhealing wounds.¹⁴

Therefore, an attractive approach for therapeutic development for chronic wounds is to focus on inhibition of these highly active proteases to restore balance between ECM remodeling and degradation.^{6,11} Peptide based inhibitors of proteases are attractive as they are derived from naturally occurring amino acids. In addition, specific peptides can offer inhibitory activity against specific proteases. However, there are downfalls when using peptides. Some of these shortcomings are their vulnerability to being destroyed quickly¹⁵ as well as their diffusional properties causing them to quickly leave the application site and spread into other tissues and areas of the body.¹⁶ This leads to the need for a delivery method that provides diffusional control and protection against degradation.

Peptides have shown promise as drug delivery platforms due to being easily editable⁴ and some noted for repetitive subunits lending an inherent ability to self-assemble.¹⁷ Elastin-like-Peptides (ELPs) offer an attractive drug delivery platform due to their unique phase transition property.¹⁸ Since they are genetically encodable, fusion proteins containing the peptide to be delivered can be synthesized using standard cloning approaches.^{19,20} The inventors have previously shown that these fusion proteins retain the phase transition properties of ELP as well as the activity of the fused protein.^{21,22} The phase transition properties of ELPs allow them to self-assemble and aggregate at body temperature, which may provide prolonged presence in the application site¹⁶ as well as the opportunity to create larger bioactive materials.²³ Thus, ELPs have proven to be useful not only for protein purification but also as bioactive peptide delivery platforms.^{16,17,24}

Inhibiting tissue breakdown is absolutely key in pursuing closure of a delayed wound and can be done easily by targeting secreted proteases that are commonly found at high levels in chronic wounds. Tissue construction is heavily reliant on the secretion of protein growth factors which contain an important role in cell migration, proliferation, and cellular matrix secretion¹⁷. However, the same proteases that break down wounded tissue, also wreak havoc on any proteins present in the site¹⁸, which includes the very important growth factors needed to remodel. The prior will keep the wound halted in a deconstruction phase. Without the necessary growth factors present, cellular migration and proliferation will happen on a very low level if not at all. The proteases present in chronic wounds destroy healthy tissue and viable signaling proteins, ultimately halting the normal wound healing process in a vicious loop that only focuses on deconstructing the wound site.

Given the shortcomings of current chronic wound therapies as well as the difficulties in effectively treating chronic wounds, the inventors have developed a multifunctional protease inhibitor capable of inhibiting different proteases with one therapeutic. The inventors have also used this multifunctional protease inhibitor as a part of a system in conjunction with conditioned media and tethering peptides to allow for sustained release of the therapeutic agents at the wound site.

### SUMMARY OF INVENTION

Natural wound healing processes allude to potential biologics that can impede the chronic breakdown of tissue, while restoring deposition of new tissue, and effectively leading to a healed wound. Proteases secreted by the body's immune system lay waste to even the healthy tissues in wounds, which can be seen in those that turn chronic, as a progressive and persistent break down of tissue, proteins, and cells in the wound site. The disruption of this exacerbated tissue breakdown is both essential to quell the destruction of healthy tissues as well as other proteins such as growth factors that are needed to signal for the development and deposition of new tissue. The latter points to the importance of the balance between tissue break down and deposition, commonly referred to as remodeling, as a focusing point for the healing of chronic wounds. The two generalized secreted molecules that balance tissue breakdown and deposition are proteases and growth factors respectively. However, the efficacy of growth factors that exist in a wound site are highly dependent upon the concentration of secreted proteases. Therefore, in chronic wounds the proteases which are continuously secreted, without intrinsic inhibition, will chew up the growth factors responsible for signaling deposition, which causes tissue destruction to be the overpowering and vicious result.

A novel multifunctional protease inhibitor was constructed to exhibit a single molecular platform containing a simultaneous multifunctionality through proper spacing given by an Elastin-like Peptide (ELP) backbone centered between the two differing inhibitory domains. This approach allows construction of proteins on a single molecular platform which can be added to, rather than constructing inhibitors. This allows for the possibility to use one bioreactor to create a protein with multiple functions thus minimizing the cost to produce.

This novel multifunctional protease inhibitor is combined with conditioned media from stem cells and tethering peptides to comprise a protein-based combination therapeutic agent capable of rebalancing tissue breakdown and deposition to tend toward minimal deconstruction with overpowering construction. The composition is capable of inhibiting proteases, flooding the site with many growth factors, while also sustaining the presence of the treatment over longer periods to avoid site disturbance and leakage of the biologic combination into neighboring tissues. The therapeutic agent is a potent influencer of cellular division by taking advantage of stem cell by-products while inhibiting proteases that break down both the healthy tissue and growth factors and providing peptides needed to encourage cellular division and the deposition of a new ECM.

The molecular tethering peptide of the therapeutic agent is used to control aggregation and provide sustained, local, delivery of the recombinant protease inhibitor. The tethering peptide was constructed to add to the dry composition, containing the conditioned supernatant and the recombinant protease inhibitor, to sustain the release and presence of the protease inhibitor proteins local to the application site. This tethering peptide acts to hold the multifunctional protease inhibitor close to extracellular matrix (ECM) components that are degraded by proteases-components that are essential for cellular migration, division, and healthy establishment. The easy editability of recombinant proteins encoded and expressed in microorganisms allows for further targeting of possible bacterial proteases that are present during infection and degrade essential tissues and growth factors for bacterial nutrients.

This advanced wound healing therapeutic is a powder-based, freeze-dried therapeutic that can be used in powder form or reconstituted into numerous different mediums or materials for desired application and results. Additionally, the design of specific protocols for product production, mixing, application, and testing has been established and optimized to make the most potent and effective product as possible, while maintaining quality control and assurance throughout. This powder composition is a self-protecting wound healing system that directly solves imbalances in the remodeling process, while being easily produced, stored, and applied. Furthermore, dry powder can easily be applied in harsh environments such as warzones, underserved communities, and even in other mammals like pets and livestock where fast wound healing is essential for better outcomes and infection minimization that may lead to chronicity and sepsis leading to severe health problems including death.

In an embodiment, a composition is presented comprising: conditioned media from stem cells; at least one multifunctional protease inhibitor; and at least one tethering peptide. The composition may further comprise at least one antimicrobial fusion peptide. The composition may be in the form of a dry acellular mixture.

The stem cells may be mesenchymal stem cells, or more specifically, mesenchymal stem cells derived from the umbilical cord. The conditioned media may comprise at least one growth factor.

The at least one multifunctional protease inhibitor may be comprised of an elastase inhibitor attached to one end of an elastin-like peptide (ELP) with a matrix metalloproteinase inhibitor (MMPI) attached to an opposing end of the ELP. The elastase inhibitor may be PARS intercerebralis major peptide D2 (PMP-D2). The MMPI may be β-amyloid precursor protein-derived inhibitory peptide (APP-IP). The ELP of the multifunctional protease inhibitor may be L10flag.

The tethering peptide may comprise an extracellular matrix (ECM) peptide attached to an elastin-like peptide (ELP). The ECM peptide may be placental growth factor 2 (PlGF2). The ELP of the tethering peptide may be L10flag.

In an embodiment, a method of treating a wound in a patient in need thereof is presented comprising: administering to the patient a therapeutically effective amount of a composition comprising conditioned media from stem cells wherein the conditioned media comprises at least one growth factor; at least one multifunctional recombinant protease inhibitor; and at least one tethering peptide wherein administration of the composition enhances wound healing to treat the wound. The composition may further comprise at least one antimicrobial fusion peptide. The composition may be in the form of a dry acellular mixture.

The stem cells may be mesenchymal stem cells, or more specifically, mesenchymal stem cells derived from the umbilical cord. The conditioned media may comprise at least one growth factor.

The at least one multifunctional protease inhibitor may be comprised of an elastase inhibitor attached to one end of an elastin-like peptide (ELP) with a matrix metalloproteinase inhibitor (MMPI) attached to an opposing end of the ELP. The elastase inhibitor may be PARS intercerebralis major peptide D2 (PMP-D2). The MMPI may be β-amyloid precursor protein-derived inhibitory peptide (APP-IP). The ELP of the multifunctional protease inhibitor may be L10flag.

The tethering peptide may comprise an extracellular matrix (ECM) peptide attached to an elastin-like peptide (ELP). The ECM peptide may be placental growth factor 2 (PlGF2). The ELP of the tethering peptide may be L10flag.

In an embodiment, a kit for treating a wound is presented comprising: a dry composition comprising conditioned media from stem cells; at least one multifunctional protease inhibitor; and at least one tethering peptide; and instructions for use. The composition may further comprise at least one antimicrobial fusion peptide. The composition may be in the form of a dry acellular mixture. The kit may further comprise a fibrin gel.

The at least one multifunctional protease inhibitor may be comprised of an elastase inhibitor attached to one end of an elastin-like peptide (ELP) with a matrix metalloproteinase inhibitor (MMPI) attached to an opposing end of the ELP. The elastase inhibitor may be PARS intercerebralis major peptide D2 (PMP-D2). The MMPI may be β-amyloid precursor protein-derived inhibitory peptide (APP-IP). The ELP of the multifunctional protease inhibitor may be L10flag.

The tethering peptide may comprise an extracellular matrix (ECM) peptide attached to an elastin-like peptide (ELP). The ECM peptide may be placental growth factor 2 (PlGF2). The ELP of the tethering peptide may be L10flag.

In an embodiment, a method of minimizing rejection of an implanted material into a patient is presented comprising: incorporating or having incorporated a dry composition into the implanted material during a manufacturing process of the implanted material, the dry composition comprising conditioned media from stem cells; at least one multifunctional protease inhibitor; and at least one tethering peptide; and implanting or having implanted the implanted material into the patient wherein the dry composition induces cellular proliferation in implant area and minimizes rejection of the implanted material by masking the implanted material with the conditioned media products that do not elicit an immune response.

The at least one multifunctional protease inhibitor may be comprised of an elastase inhibitor attached to one end of an elastin-like peptide (ELP) with a matrix metalloproteinase inhibitor (MMPI) attached to an opposing end of the ELP. The elastase inhibitor may be PARS intercerebralis major peptide D2 (PMP-D2). The MMPI may be β-amyloid precursor protein-derived inhibitory peptide (APP-IP). The ELP of the multifunctional protease inhibitor may be L10flag.

The tethering peptide may comprise an extracellular matrix (ECM) peptide attached to an elastin-like peptide (ELP). The ECM peptide may be placental growth factor 2 (PlGF2). The ELP of the tethering peptide may be L10flag.

In a further embodiment, a multifunctional protease inhibitor is presented comprising: at least two bioactive molecules; and at least one elastin-like peptide (ELP) bound at each end to one of the at least two bioactive molecules. One of the at least two bioactive molecules may be an elastase inhibitor such as PARS intercerebralis major peptide D2 (PMP-D2). One of the at least two bioactive molecules may be a matrix metalloproteinase inhibitor (MMPI), such as β-amyloid precursor protein-derived inhibitory peptide (APP-IP). The ELP may be L10flag.

### BRIEF DESCRIPTION OF THE DRAWINGS

For a fuller understanding of the invention, reference should be made to the following detailed description, taken in connection with the accompanying drawings, in which:

FIG. 1A-B are a series of images depicting inverse temperature cycling results in successful purification of the hybrid dual protease inhibitor. After protein expression of PMPD2-ELP-APP-IP in BLR(DE3) cells, the cells were then lysed, and the protein was purified using inverse temperature cycling. (a) A total protein stain was performed by running the samples down an SDS-PAGE gel then transferring the samples to a nylon membrane, which was then exposed to Coomassie Blue G250 reagent. The subsequent total protein stain displays in Lane 1 of the figure, a biotinylated ladder; lane 2, a sample of the lysate; and lane 3, a sample of the final purified protein. (b) A western blot was then performed with specificity to PMPD2-ELP-APP-IP's FLAG-tag region. Lane 1: biotinylated ladder and Lane 2: purified protein. The biotinylated ladder was detected using anti-biotin HRP-linked antibody, while the target protein was detected utilizing Anti-FLAG (Rabbit) primary antibody and anti-Rabbit IgG HRP-linked secondary antibody. The mass of the purified protein is 26.8 kDa, which the protein's band aligns with the corresponding band of the biotinylated ladder in the corresponding image.

FIG. 2A is an image depicting the dual protease inhibitor inhibits neutrophil elastase (NE) like NE inhibitor, PMPD2-ELP. A kinetic colorimetric NE inhibition assay was performed to evaluate various concentrations of (a) PMPD2-ELP-APP-IP.

FIG. 2B is an image depicting the dual protease inhibitor inhibits neutrophil elastase (NE) like NE inhibitor, PMPD2-ELP. A kinetic colorimetric NE inhibition assay was performed to evaluate various concentrations of (b) PMPD2-L10-ELP.

FIG. 2C is an image depicting the dual protease inhibitor inhibits neutrophil elastase (NE) like NE inhibitor, PMPD2-ELP. (c) From the kinetic inhibition assays, slope (activity) was calculated for each protein concentration and 1/Activity versus 1/[protein concentration] was plotted.

FIG. 3A is an image depicting the dual protease inhibitor inhibits Matrixmetalloproteinase-2 (MMP-2) like MMP2 inhibitor, APP-IP-ELP. A kinetic colorimetric MMP-2 inhibition assay was performed to evaluate inhibitory activity of various concentrations of (a) PMPD2-ELP-APP-IP.

FIG. 3B is an image depicting the dual protease inhibitor inhibits Matrixmetalloproteinase-2 (MMP-2) like MMP2 inhibitor, APP-IP-ELP. A kinetic colorimetric MMP-2 inhibition assay was performed to evaluate inhibitory activity of various concentrations of (b) APP-IP-ELP.

FIG. 3C is an image depicting the dual protease inhibitor inhibits Matrixmetalloproteinase-2 (MMP-2) like MMP2 inhibitor, APP-IP-ELP. (c) From the kinetic inhibition assays, slope (activity) was calculated for each protein concentration and 1/Activity versus 1/[protein concentration] was plotted.

FIG. 4 is an image depicting the dual protease inhibitor is not toxic to cells at its active concentrations. A549 cells were treated with 0.05, 0.2, and 0.5 μg PMPD2-ELP-APP-IP per μl of cell culture media and a control consisting of cells with no protein treatment. A cell viability Hoechst assay was performed and the OD values for each experimental group was normalized to the control to evaluate % viability.

FIG. 5 is an image depicting the dual protease inhibitor protects Keratinocyte Growth Factor (rhKGF) from both NE and MMP-2. rhKGF was incubated with either purified HNE or MMP-2 for 2, 4, or 24 h. Proper experimental controls were established by incubating KGF with HNE or MMP-2 without the dual protease inhibitor, PMPD2-ELP-APP-IP, to display the degree of KGF degradation. At the indicated time points samples were taken and subjected to western blots using an antibody specific to human KGF to evaluate the effectiveness of the dual protease inhibitor in protecting KGF from proteolytic degradation.

FIG. 6 is an image depicting the wound healing system described herein.

FIG. 7A-B are a series of images depicting (A) the Proliferation of C2C12 Cells Treated with Various Collections of Um-MSC Conditioned Media.*Statistically significant compared to an untreated control with p≤0.05 and F_{crit}≤F. Liquid MSC-CM Application. (B) the Degradation of MSC Conditioned Products by Various Concentrations of NE. Elastase Concentrations: 200, 20, 2, 0.2, 0.02 mU/mL. *Statistically significant when compared to sample MSC-CM/0 NE, p≤0.05 and F_{crit}≤F. MSC-CM Sample 3 from Figure A. Liquid MSC-CM Application. The MSC Conditioned Media Sample number 3 from (A) was degraded by various neutrophil elastase concentrations.

FIG. 7C is a graph depicting FIG. 23. Um-MSC Conditioned Media in Various Treatment Forms Tested for Inducing Proliferation of C2C12 Cells. *Statistically significant with p≤0.05 and F_{crit}≤F when compared to the untreated control.

FIG. 8 is a Western blot depicting total Protein Stain of Samples of L10Flag-PlGF2 and PMPD2-L10Flag for the Evaluation of Affinity of L10F-PlGF2 for Various Matrix Proteins.

FIG. 9A-C are a series of images depicting ELP-Fusion Protein Transitioning Behavior for A) Unfused ELPs, B) Single ELP Fusions, and C) Dual ELP Fusions.

FIG. 10 is an image depicting transitioning behavior of dual ELP fusions utilizing pI-based phase transitioning.

FIG. 11A-B are a series of graphs depicting the Degree of Transitioning of L10Flag and PMPD2-L10Flag-APPIP a Neutral pH and at the Protein's Isoelectric Point. A) L10Flag at a neutral pH and its pI. B) PMPD2-L10Flag-APPIP at a neutral pH and its pI.

FIG. 12A-B are a series of images depicting Microscopy Imaging of Transitioned PMPD2-L10Flag-APPIP. A) At PMPD2-L10Flag-APPIP's pI B) PMPD2-L10Flag-APPIP at a neutral pH.

FIG. 13A-C are a series of graphs depicting Dynamic Light Scattering Data for L10Flag at a 7.4 pH, from 10° C. to 30° C. L10Flag at a neutral pH over temperatures A) 10° C., B) 30° C., and C) 37° C.

FIG. 14A-C are a series of graphs depicting Dynamic Light Scattering Data for PMPD2-L10Flag-APPIP at a 7.4 pH, from 10° C. to 40° C. A) PMPD2-L10Flag-APPIP a neutral pH over temperatures A) 10° C., B) 37° C., and C) 40° C.

FIG. 15A-C are a series of graphs depicting Dynamic Light Scattering Data for PMPD2-L10Flag-APPIP at its pI, 4.1 pH, from 10° C. to 40° C. PMPD2-L10Flag-APPIP at its pI over temperatures A) 10° C., B) 37° C., and C) 40° C.

FIG. 16A-B are a series of images depicting (A) mice wounds from the NE pretreated controls taken at the experiment's end point; (B) mice wounds from the experimental groups that were NE pretreated and then received MSC conditioned media treatments with continuous NE treatments.

FIG. 16C-D is a series of images depicting (C) mice wounds from the experimental groups that were NE pretreated and then received MSC conditioned media and PMPD2-L10Flag-APP with continuous NE treatments and (D) mouse treated with MSC-CM and dual inhibitor with would packed with the therapeutic powder.

FIG. 17A-C are a series of representative images of Masson's Trichrome Stained Wound Tissue Samples. Utilized for Collagen and Epidermal Thickness Quantifications Where A) is a Sample from the Untreated Control, B) is from the Treatment with MSC-CM, and C) Received the Full Treatment, MSC-CM and PMPD2-L10Flag-APPIP, Where the Dashed Line Represents the Basement Membrane, and E: Epidermis, D: Dermis.

FIG. 18 is an image depicting the average Epidermal Thickness from Tissue Samples Collected from Each Mouse Group Measured in ImageJ and Normalized to the Untreated Control. *Statistically significant when compared to the untreated control group with p≤0.05 and F_{crit}≤F.

FIG. 19 is an image depicting collagen Composition of the Mice Wounds Normalized to the Collagen Composition of the Untreated Control. *Statistically significant with p≤0.05 and F_{crit}≤F.

FIG. 20 is a series of images depicting Flow Cytometry Results of Um-MSC's Targeting the Expression of Each Positive Marker for Mesenchymal Stem Cell Determination.

FIG. 21 is a series of images depicting Flow Cytometry Results of Fully Stained Cells Without DAPI to Determine the Degree of Negative Marker Expression.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

All numerical designations, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined. As used herein, the term “about” refers to ±10% of the numerical.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used in the specification and claims, the singular form “a”, “an” and “the” include plural references unless the context clearly dictates otherwise. For example, the term “a nanoparticle” includes a plurality of nanoparticles, including mixtures thereof.

As used in this specification and the appended claims, the term “or” is generally employed in its sense including “and/or” unless the context clearly dictates otherwise.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance. “Consisting of” shall mean excluding more than trace elements of other components or steps.

As used herein “patient” is used to describe a mammal, preferably a human, to whom treatment is administered, including prophylactic treatment with the compositions of the present invention. Non-limiting examples of mammals include humans, rodents, aquatic mammals, domestic animals such as dogs and cats, farm animals such as sheep, pigs, cows and horses. “Patient” and “subject” are used interchangeably herein.

“Pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. In some embodiments, the pharmaceutically acceptable carrier is a gel, ointment, hydrogel, cream, aerosol, or powder. Formulations are described in a number of sources that are well known and readily available to those skilled in the art. For example, Remington's Pharmaceutical Sciences (Martin E W Easton Pennsylvania, Mack Publishing Company, 19^{th} ed.) describes formulations which can be used in connection with the subject invention.

The terms “administer” or “administering” as used herein are defined as the process by which the compositions of the present invention are delivered to the patient for treatment or prevention purposes. The composition can be delivered topically in dosage unit formulations containing conventional nontoxic pharmaceutically acceptable carriers, adjuvants, and vehicles as desired. Administration may occur once or multiple times. Non-limiting examples of topical administration include, but are not limited to, a powder, gel, hydrogel, aerosol, cream, transdermal patch, spot-on treatment, or ointment.

“Sustained release” as used herein refers to a composition comprising a therapeutically effective amount of the composition, when administered to a patient, continuously releases a stream of one or more active agents over a predetermined time period at a level sufficient to achieve a desired effect throughout the predetermined time period. Reference to a continuous release stream is intended to encompass release that occurs as the result of biodegradation of the composition, or component thereof, or as the result of metabolic transformation or dissolution of the added nutrients or other desired agents.

A “therapeutically effective amount” as used herein is defined as concentrations or amounts of components which are sufficient to effect beneficial or desired clinical results, including, but not limited to, any one or more of treating or preventing symptoms of a wound, including chronic wounds, or implant rejection.

The dosing of compounds and compositions to obtain a therapeutic or prophylactic effect is determined by the circumstances of the patient, as is known in the art. The dosing of a patient herein may be accomplished through individual or unit doses of the compounds or compositions herein or by a combined or prepackaged or pre-formulated dose of a compounds or compositions.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current invention according to the foregoing considerations.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequently. Administration can also be continuous and adjusted to maintaining a level of the compound within any desired and specified range.

The administration of the composition can be extended over an extended period of time, such as from about a week or shorter up to about a year or longer. For example, the dosing regimen can be extended over a period of any of about 2, 3, 4, 5, 6, 7, 8, 9, 10, 11, and 12 months. In some embodiments, there is no break in the dosing schedule. In some embodiments, the interval between each administration is no more than about a week.

The compounds used in the present invention may be administered individually, or in combination with or concurrently with one or more other compounds used to promote wound healing. Additionally, compounds used in the present invention may be administered in combination with or concurrently with other therapeutics to prevent implant rejection.

“Prevention” or “preventing” as used herein refers to any of: halting the effects of a wound or implant rejection, reducing the effects of a wound or implant rejection, reducing the incidence of a wound or implant rejection, reducing the development of a wound or implant rejection, delaying the onset of symptoms of a wound or implant rejection, increasing the time to onset of symptoms of a wound or implant rejection, and reducing the risk of development of a wound or implant rejection.

“Treatment” or “treating” as used herein refers to any of: the alleviation, amelioration, elimination and/or stabilization of a symptom, as well as delay in progression of a symptom of a particular disorder. For example, “treatment” may include any one or more of the following: amelioration and/or elimination of one or more symptoms associated with a wound or implant rejection, reduction of one or more symptoms of a wound or implant rejection, stabilization of symptoms of a wound or implant rejection, and delay in progression of one or more symptoms of a wound or implant rejection.

“Fusion peptide” or “fusion protein” as used herein refers to a peptide in which at least one bioactive molecule is attached to a polypeptide backbone. In some embodiments, the polypeptide backbone is comprised of elastin-like peptides (ELPs). The multifunctional recombinant protease inhibitor and tethering peptide are both considered fusion peptides.

“Bioactive molecule” as used herein refers to a peptide capable of exerting a beneficial biological effect on a wound of a patient. The bioactive molecule may be attached to an ELP to form a fusion peptide. Bioactive molecules include, but are not limited to, ECM binder peptides, protease inhibitor peptides such as MMP resistant peptides and elastase resistant peptides, and antimicrobial peptides.

“Elastin-like peptide (ELP)” as used herein refers to biodegradable, non-immunogenic protein-based polymers composed of tandemly repeated blocks of (Val-Pro-Gly-X-Gly)n (SEQ ID NO:1) where X can be any residue but Pro and n is the number of repeated blocks (length of the ELP). In some embodiments, the ELP is L10FLAG.

“Elastase resistant peptide” or “elastase inhibitor” as used herein refers to a peptide which is shown to inhibit neutrophil elastase (HNE) in chronic wounds. Examples of elastase resistant peptides include, but are not limited to, PMP-D2.

“Matrix metalloproteinase (MMP) resistant peptide” or “matrix metalloproteinase inhibitor (MMPI)” as used herein refers to a peptide which is shown to inhibit MMPs in chronic wounds. Several MMP resistant peptides are contemplated for use in the instant invention including, but not limited to, APP-IP.

“Multifunctional protease inhibitor” or “multifunctional recombinant protease inhibitor” as used herein refers to a single recombinant protein capable of inhibiting different specific proteases and having at least 2 different bioactive molecules, each represented by a different protease inhibitor, separated by an ELP. The multifunctional protease inhibitor is a single molecule with the capabilities to inhibit specific proteases without the mixing of multiple components. It provides the potential to add additional inhibitors with specificity to unique proteases leading to a therapy that can easily be tailored to a unique chronic wound environment. In some embodiments, the multifunctional protease inhibitor is a dual protease inhibitor having the general formula of protease inhibitor 1-ELP-protease inhibitor 2. In some embodiments, protease inhibitor 1 may comprise an elastase inhibitor while protease inhibitor 2 may comprise an MMPI. Other configurations of multifunctional protease inhibitor are contemplated with each bioactive molecule being a different protease inhibitor separated by an ELP.

“Extracellular matrix (ECM) binder peptide” as used herein refers to a bioactive molecule capable of binding to the extracellular matrix components such as collagen, elastin, fibrin, fibronectin, fibrinogen, etc. Examples of ECM binder peptides include, but are not limited to, placental growth factor 2 (PlGF2).

“Tethering peptide” or “tethering protein” as used herein refers to a fusion peptide having a bioactive molecule that is capable of binding to the extracellular matrix (ECM binder peptide) attached to one end of an ELP with the opposing end of the ELP being free to attach to an ELP of a separate fusion peptide, such as a multifunctional protease inhibitor or an antimicrobial fusion peptide, to tether that separate fusion peptide to the extracellular matrix to permit sustained release.

“Antimicrobial peptide (AMP)” as used herein refers to a peptide which acts to have a beneficial biological effect on a microbial infection. Examples of an antimicrobial functional peptide derived from mammals for use in the instant invention include, but are not limited to, SR-0379; defensins such as α-defensin 1, α-defensin 4, α-defensin 5, α-defensin 6, β-defensin 1, β-defensin 2, β-defensin 3, β-defensin 4, antifungal heliomycin, defensin-like peptide-2, and sugarcane defensin 5; cathelicidins such as BMAP-27, LL-37, fowlicidin-1, fowlicidin-2, fowlicidin-3, protegrin-3, and protegrin PG-5. While examples of mammalian-derived antimicrobial functional peptides are given, the invention contemplates all antimicrobial peptides whether synthetic or naturally derived.

“Antimicrobial fusion peptide” or “antimicrobial ELP fusion peptide” as used herein refers to a fusion peptide having a bioactive molecule that is capable of having a beneficial biological effect on a microbial infection (antimicrobial peptide) attached to an ELP.

A “beneficial biological effect” as used herein refers to exhibition of an effect that is associated with wound healing or inhibition of implant rejection. Examples of beneficial biological effects include, but are not limited to, re-epithelialization, granulation, angiogenesis, upregulation of collagen production, absence of or improvement of infection, etc.

“Conditioned media” or “conditioned supernatant” as used herein refers to culture media containing biologically active components secreted by cells that were previously cultured in the media and removed. Such biologically active components include growth factors, metabolites, and extracellular matrix proteins. In some embodiments, the previously cultured cells are stem cells. In some embodiments, the stem cells are mesenchymal stem cells, however all types of stem cells are contemplated for use herein.

“Growth factor” as used herein refers to substances secreted by the body that function to stimulate the growth and proliferation of the cells involved in wound healing and inflammation thus resulting in faster wound healing. In some embodiments, the growth factors are from a conditioned media from stem cells such as mesenchymal stem cells (MSCs). Examples of growth factors that may be used herein include, but are not limited to: epidermal growth factor (EGF); keratinocyte growth factor (KGF); transforming growth factor (TGF); vascular endothelial growth factor (VEGF) including BMP-2; platelet-derived growth factor (PDGF); fibroblast growth factor (FGF), including basic fibroblast growth factor (bFGF); insulin-like growth factors (IGF-1 and IGF-2); hepatocyte growth factor (HGF); vascular cell adhesion molecule (VCAM); macrophage stimulating protein (MSP); interleukins (IL); colony-stimulating factors (CSF) and combinations thereof.

“Chronic wound” as used herein refers to a wound which lingers in the inflammatory phase of wound healing without advancing to the proliferative phase and is thus unable to heal. Wounds such as diabetic, venous or decubitus ulcers are considered to be chronic wounds. The term “wound” as used herein refers to injuries to living tissue. In some embodiments, “wound” refers to injuries to the skin.

The body's immune system and cellular response are responsible for the breakdown of damaged tissue through the secretion of proteinases that target the tissues structural components and delay healing⁶. This is a complex process that involves different cell types and their associated protease secretions, but it is well known what secretions are responsible for the breakdown of different tissue types like neutrophil elastase (NE) and matrix metalloproteinase-2 (MMP2). An added aspect of tissue degradation in chronic and HtHWs is an established bacterial infection leading to further proteolytic degradation^{36, 6, 37} Bacteria secrete their own metalloproteinases to aid in a competitive establishment and acquire the nutrients necessary to expand and take over a wound site^{5,7}. However, this displays commonality across two different contributing factors to HtHWs. That is in one case it is our body oversecreting tissue degrading enzymes, and in the case of bacteria they also secrete tissue degrading proteases to survive, grow, and compete. The latter draws importance to the fact that it does not matter what the case is, the result is tissue breakdown. Inhibiting tissue breakdown is absolutely key in pursuing closure of a delayed wound and can be done easily by targeting secreted proteases that are commonly found at high levels in chronic wounds. Tissue construction is heavily reliant on the secretion of protein growth factors which contain an important role in cell migration, proliferation, and cellular matrix secretion³⁹. However, the same proteases that break down wounded tissue, also wreak havoc on any proteins present in the site¹², which includes the very important growth factors needed to remodel. The prior will keep the wound halted in a deconstruction phase. Without the necessary growth factors present, cellular migration and proliferation will happen on a very low level if not at all. The proteases present in chronic wounds destroy healthy tissue and viable signaling proteins, ultimately halting the normal wound healing process in a vicious loop that only focuses on deconstructing the wound site.

Chronic and hard to heal wounds cause long term pain, financial burden, and are subject to antibiotic resistant infection if not closed in realistic time frames. Current treatments have their usefulness, such as debridement to avoid issues like infection or urea treatments for protease inhibition, however each strategy is employed separately and must be reapplied consistently to observe results. Regenerative medicine products for tissue regeneration generally are quite expensive. The majority of the products claim to be “live” cellular products, however they contain few actual live cells and do not exhibit much therapeutic value, if any. What live cells are present in the products easily die under freezer storage or during shipping. Furthermore, current strategies lack a targeted therapeutic approach.

In light of the shortcomings of the prior art, the inventors have developed compositions, system and associated methods to assist in decreasing the time necessary for wound closure as well as minimizing risk of infection or further tissue degradation beyond what is needed for remodeling. Protease inhibitors can be used to halt the proteolytic degradation of tissue and associated growth factors to progress the wound towards closure. While protease inhibitors can protect the tissue and associated growth factors from degradation, the addition of both protease inhibitors and additional growth factors can be beneficial in accelerating wound closure.

In Example 1, an exemplary multifunctional protease inhibitor is presented which is capable of inhibiting two different proteases with administration a single protein. In Example 2, a system/composition is presented which utilizes the multifunctional protease inhibitor of Example 1 in combination with conditioned media and a tethering peptide to form a composition that can be used as a shotgun approach or tailored to a specific wound environment depending on patient/clinic type, application environment, etc.

The following non-limiting examples illustrate exemplary compositions, systems and methods thereof in accordance with various embodiments of the disclosure. The examples are merely illustrative and are not intended to limit the disclosure in any way.

### Example 1—Multifunctional Protease Inhibitor (Dual Protease Inhibitor)

A peptide-based inhibitor was reported to impede the activity of two proteases that are highly active in chronic wounds namely neutrophil elastase (HNE)^{12,25} and Matrix metalloprotease-2 (MMP-2).^{5,10} Two peptides, PMPD2¹² and APP-IP,²⁶ block HNE and MMP-2,respectively, and were fused to each end of an ELP. The objective was to create a single protein that can maintain phase transitioning properties of an ELP for the utility of sustained release, while exhibiting multifunctional bioactivity. Although it is shown here for protease inhibitors, this strategy can be broadened to incorporate two molecules targeting two different processes into a single molecule for targeted delivery of combination treatments.

The high protease environment in chronic wounds presents a significant challenge in the development of therapeutics for healing. The imbalance of proteases not only results in the disruption of balance between ECM degradation and remodeling,⁸⁻¹¹ but also in the loss of other beneficial proteins such as growth factors that initiate the healing process. This further confounds treatment approaches as growth factor supplementation is considered beneficial for chronic wound treatment is rendered ineffective because of the proteases. Hence, protease inhibitors play a crucial role for the development of a successful therapy for chronic wound healing. However, this strategy is challenging due to the presence of many unique proteases present in chronic wounds.

Described here is the development of a dual protease inhibitor PMPD2-ELP-APP-IP, which consists of peptide inhibitor domains PMPD2 (HNE inhibitor) and APP-IP (MMP inhibitor). The hybrid peptide inhibitor successfully inhibited two commonly found proteases in chronic wounds namely HNE and MMP thereby eliminating the need of two individual inhibitors of the two proteases.

The IC₅₀ of the dual inhibitor was comparable with the IC₅₀ of the individual inhibitors PMPD2-ELP (For HNE) and APP-IP-ELP (For MMP). This shows that the two peptide inhibitor domains do not interfere with each other's activity. Furthermore, the specificity of the domains is maintained in the fusion as PMPD2 did not significantly inhibit MMP-2 and APP-IP did not significantly inhibit HNE. These data indicate that the hybrid fusion protein is useful as it inhibits two proteases. Therefore, PMPD2-ELP-APP-IP is a single protein designed to have broad spectrum effect, just as heterogeneously mixing the two single inhibitory peptides would accomplish the same goal. However, heterogeneously mixing the two peptides would classify the treatment as a drug combination to accomplish broad-spectrum activity. The hybrid protein, PMPD2-ELP-APP-IP, can be classified as a single treatment, but with two unique functions. Avoidance of heterogeneous mixing of ELP-fusion proteins eliminates questions of drug interactions and allows for predictability of phase transitioning properties and uniformity of self-assembly that governs sustained release. Although one may think the utilization of multiple inhibitors rather than one multifunctional inhibitor is more desirable when a 1:1 mixing ratio is not the case. However, delivering a single molecule is more desirable compared to delivering two inhibitors as there could be issues related to hindrance from co-localization, concentration, and undesirable interactions with each other.

The dual protease inhibitor may be a candidate to disrupt the imbalance between ECM degradation and remodeling as its fusion with an ELP provides useful as a drug delivery platform for biologically active peptides. Proving this concept leads to the possible capability of creating additional proteins with more biologically active regions to target two or more proteases that are upregulated in chronic wounds by adding additional inhibitory peptides to the amino acid sequence. In contrast, a current treatment to inhibit proteases, such as NE, is oleic acid and albumin mixtures.²⁸ Oleic acid and albumin treatments consist of a heterogeneous mixture of two components, where this mixture is nonspecific. In contrast, PMPD2-L10FLAG-APP-IP is a single molecule with the capabilities to inhibit specific proteases without the mixing of multiple components and provides the potential to add additional inhibitors with specificity to unique proteases leading to a therapy that can easily be tailored to a unique chronic wound environment.

MMP's are key proteases in the destruction of ECM components.^{10,11} However, MMP's have been shown to destroy growth factors that contain the key role in restoring the cellular components of wounded tissue². The inventors show that PMPD2-L10FLAG-APP-IP hybrid fusion protein effectively interrupts the process of keratinocyte growth factor degradation by both MMP-2 and HNE. The latter links this dual inhibitor to the potential application of using this broad-spectrum inhibitor as a growth factor protector in chronic wounds, which broadens its role as not only a control of ECM degradation but also a regulator of tissue reconstruction. Furthermore, chronic wounds display poor vasculature leading to an impaired immune response. MMP-2 is as large contributor to vasculature break down.²⁹ Using a self-assembling protein such as PMPD2-L10FLAG-APP-IP as a thermoresponsive inhibitor of MMP-2 allows for point specific application establishing vasculature rich environment for wound closure. Ultimately, point specific therapies eliminates the need to for a systemic treatment such as an antibiotic, which could bring about undesirable affects in other areas of the body and contain delivery failure to the wound bed in the presence of poor vascularization. Following the rhetoric of poor vasculature, chronic wounds are susceptible to infections due to a lack in the ability of the tissue to undergo the chain of immune response that is proper for wound cleanout and closure, which will lead to further tissue destruction as the immune cells that are present overreact to clear the wound of infection.³⁰ To support the latter, it is understood that neutrophil elastase is secreted by neutrophils in the presence of bacterial infection due the antimicrobial properties of HNE.³¹ However, due to neutrophil elastase's ability to degrade ECM, its action during an infection will be immense as the HNE is oversecreted and not properly cleared by the tissue. PMPD2-L10FLAG-APP-IP can both inhibit MMP-2 and HNE ultimately reestablishing an environment where growth factors can act to restore proper tissue construction and vascularization.

The fusion of both inhibitor domains to an ELP retained the biological activity of both domains as well as the phase transitioning properties of ELPs. This was not surprising as previous work has shown the development of several chimeric ELP fusion proteins that retain the biological activity of the fused domain as well as the physical phase transitioning property of ELPs.^{16,24,27} However, the results obtained here are novel as compared to all previous studies involving chimeric ELP fusions since here there are two domains fused to create a single molecule with two roles. Not only was the dual fusion thermoresponsive like ELPs but also had inhibiting activities toward two different proteases. The recombinant nature of ELP sequences allowed the inventors to create these complex sequences seamlessly and the thermoresponsive properties of ELPs leads to rapid purification. It can be brought to question if an ELP is needed to create novel proteins that contain multiple domains for multiple therapeutic actions. However, the ELP may allow for domain separation, where without, it may be observed that domains with various structures and charges would interact in undesirable ways if not separated by an ELP. The ability to construct proteins with more than two biologically active domains on one or more ELP backbone can be explored. As seen herein, with the dual fusion of PMPD2 and APPIP fused around a centered ELP, neither domain's inhibitory activity was negatively affected by the other, while the ELP's remain thermoresponsive after addition of a biologically active domains, making it a fusion partner that is desirable for simple purification and a drug delivery mechanism that self-assembles into stable nanoparticles and extend the life of small biologically active peptides that would otherwise be diffusively unstable.³² In chronic wounds there exists a threat to proteolytic degradation of growth factors and the application of this study sheds light on the ability to deliver protease inhibitors and protect growth factors from degradation. Koria et al. has shown that a fusion protein, KGF-ELP, remains biologically active after ELP fusion,²⁴ which contributes to a proof of concept that similar multifunctional proteins could be constructed to contain a protease inhibitor and a growth factor, where the protease inhibitor may effectively protect the growth factor while existing on the same protein platform ultimately giving a single protein the power to act as a designer therapeutic for a formulated result. The opportunities are endless in the realm of wound healing and point specific treatments. It has also been shown that antimicrobial peptides (AMPs) contain synergy³³ and some antimicrobial domains can cause disruption of bacterial biofilm production when used with a ELP based material coating.³⁴ This further supports the combining of biologically active domains around an ELP ultimately providing broad spectrum treatments and activity amplification by using a similar construction as described herein to keep two peptides with synergy or differing activity local to one another to increase therapeutic power and coverage.

### Results

### Gene Confirmation, Expression, and Protein Purification

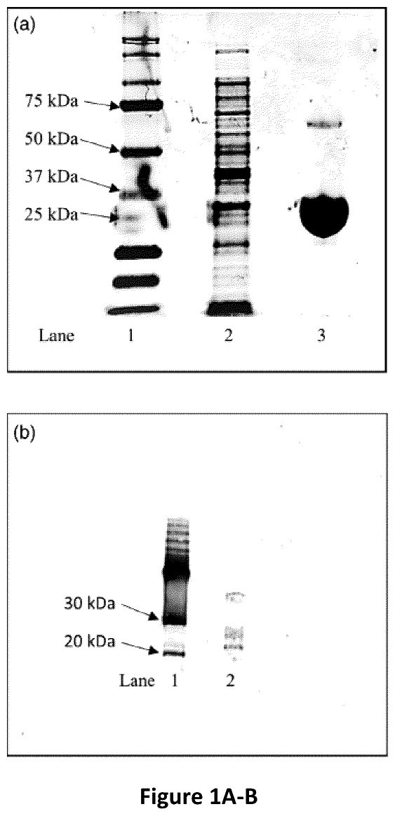

The gene encoding the fusion protein PMPD2-ELP-APP-IP was successfully cloned in the expression plasmid pET25b+using recursive directional ligation as described previously.¹⁹ The cloned gene sequence was verified by Sanger sequencing. The fusion protein was successfully expressed and purified using inverse temperature cycling. After three cycles, purified protein was obtained (FIG. 1a). The predicted molecular weight of 26.8 kDa was in line with the observed molecular weight of the fusion protein in the gel. In addition, the protein was detected and confirmed (FIG. 1b) utilizing a western blot specifically targeting the FLAG-tag region of the protein.

### Neutrophil Elastase (NE) Inhibition

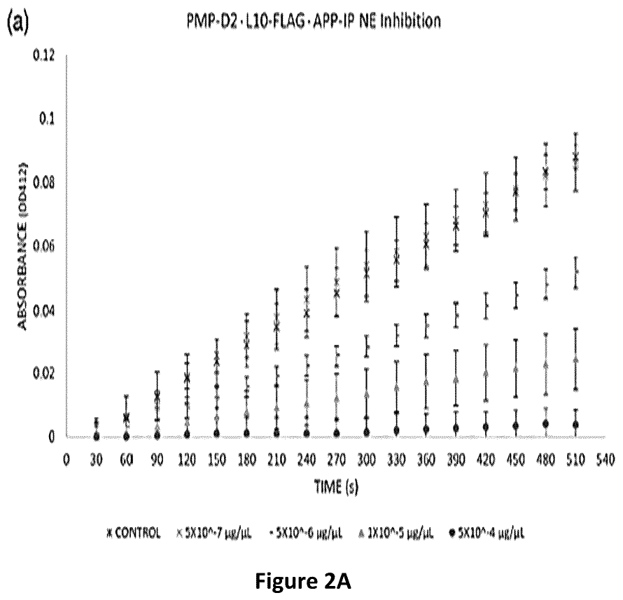

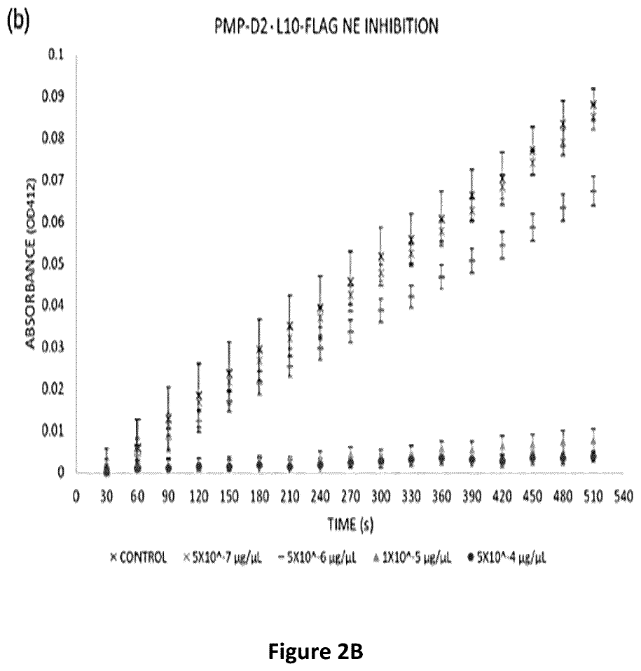

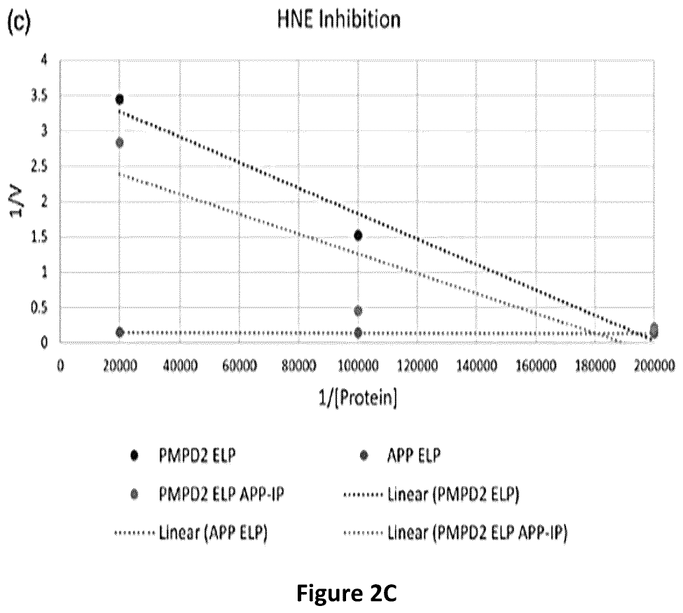

Next, the inhibition of the neutrophil elastase by the dual protease inhibitor was evaluated in order to display a maintenance of activity held by the PMPD2 domain of the protein. An enzyme-based substrate assay was used to test the inhibition of neutrophil elastase. Indeed, the dual protease inhibitor completely inhibited NE at a concentration of 5×10-⁻⁴ μg/μl (FIG. 2a). The dual protease inhibitor inhibited NE just as effectively as the single protease inhibitor PMPD2-ELP (FIG. 2b). However, as expected, APP-IP-ELP did not inhibit NE inhibition at similar concentrations. To quantitatively compare the inhibitory activity of the dual inhibitor with the single inhibitors, the IC₅₀ was evaluated for each inhibitor. The calculated IC₅₀ for the dual protease inhibitor was 8.5×10⁻⁷ μg/μl, while of the single protease inhibitor PMPD2-ELP was 7.08×10⁻⁶ μg/μl. On the other hand, APP-IP-ELP did not similarly inhibit NE demonstrating that PMPD2 is responsible for majority of NE inhibition by the dual protease inhibitor. The comparisons can be visualized in the Lineweaver-Burk plot of 1/activity versus 1/[protein] (FIG. 2c). In FIG. 2c, it can be observed that both the dual protease inhibitor and PMPD2-ELP display an increase in inhibitory activity against NE as their concentrations are increased. While APP-IP-ELP contains a near zero slope over various protein concentrations indicating insufficient activity against NE (FIG. 2c). APPIP-ELP contained 8.4% inhibition of NE near the IC₅₀ of the dual inhibitor suggesting that APP-IP domain contains some activity against NE, ultimately leading to the difference in IC₅₀ between the dual inhibitor and PMPD2-ELP.

### Matrix Metalloproteinase-2 (MMP-2) Inhibition

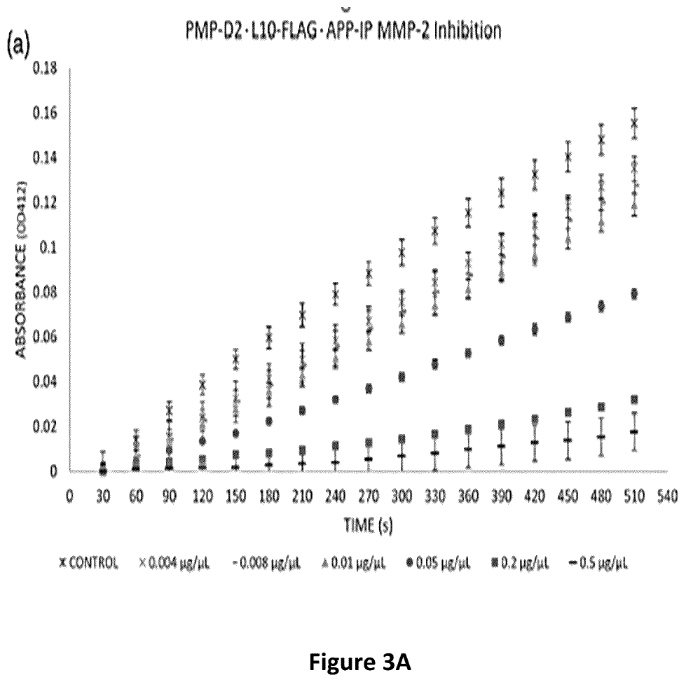

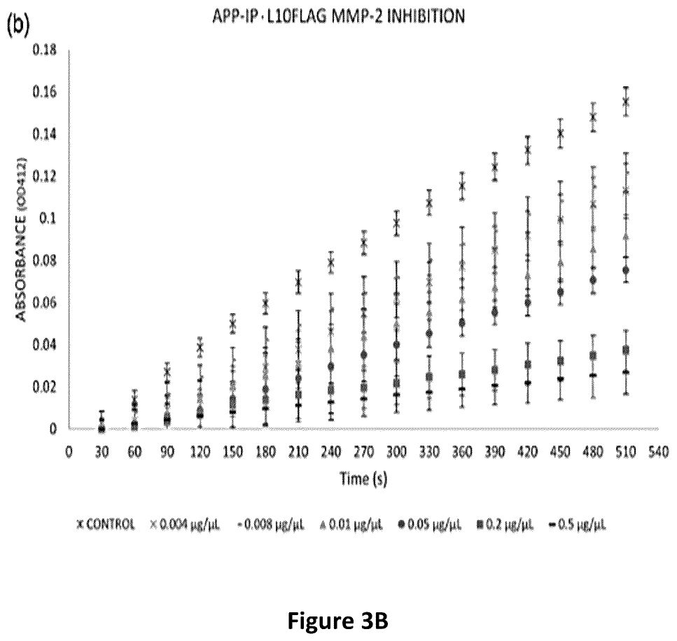

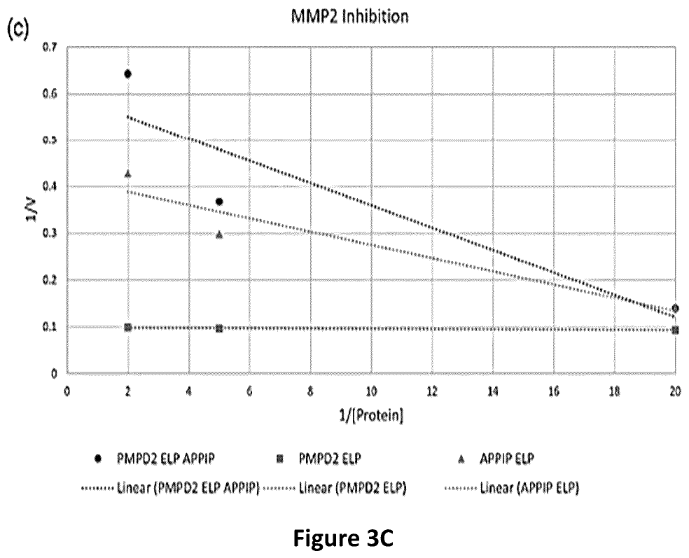

Assays were performed to ascertain whether the dual protease inhibitor is capable to inhibit MMP-2 activity. To this end, an enzyme activity substrate assays with different concentrations of the dual protease inhibitor was employed. There was near complete inhibition of MMP-2 by the dual protease inhibitor at a concentration of 0.5 μg/μl (FIG. 3a). The dual inhibitor inhibits MMP-2 activity similarly as the single MMP-2 inhibitor APP-IP-ELP across various concentrations (FIG. 3b). As expected, the single NE inhibitor PMPD2-ELP did not inhibit MMP-2 over various concentrations, indicating that PMPD2 portion in the dual protease inhibitor is not responsible for blocking the majority of the MMP-2 activity. In FIG. 3c, a Lineweaver-Burk plot of 1/activity versus 1/[protein], it can be observed that both the dual protease inhibitor and APP-IP-ELP increase their inhibitory activity against MMP-2 as concentration is increased. While PMPD2-ELP contains a near zero slope over various protein concentrations indicating insufficient activity against MMP-2 (FIG. 3c). To quantify the inhibition, the IC₅₀ of the dual inhibitor was calculated and compared with the single inhibitor. It was learned that the dual inhibitor had an IC₅₀ of 0.04 μg/μl, while the single inhibitor APP-IPELP had an IC₅₀ of 0.05 μg/μl. Moreover, the MMP-2 inhibitory activity PMPD2-ELP leveled off at 20% inhibition at the largest tested concentration indicating that it is a poor inhibitor of MMP-2 and is not the main contributor to the MMP-2 inhibition exhibited by the dual inhibitor.

### Cell Toxicity

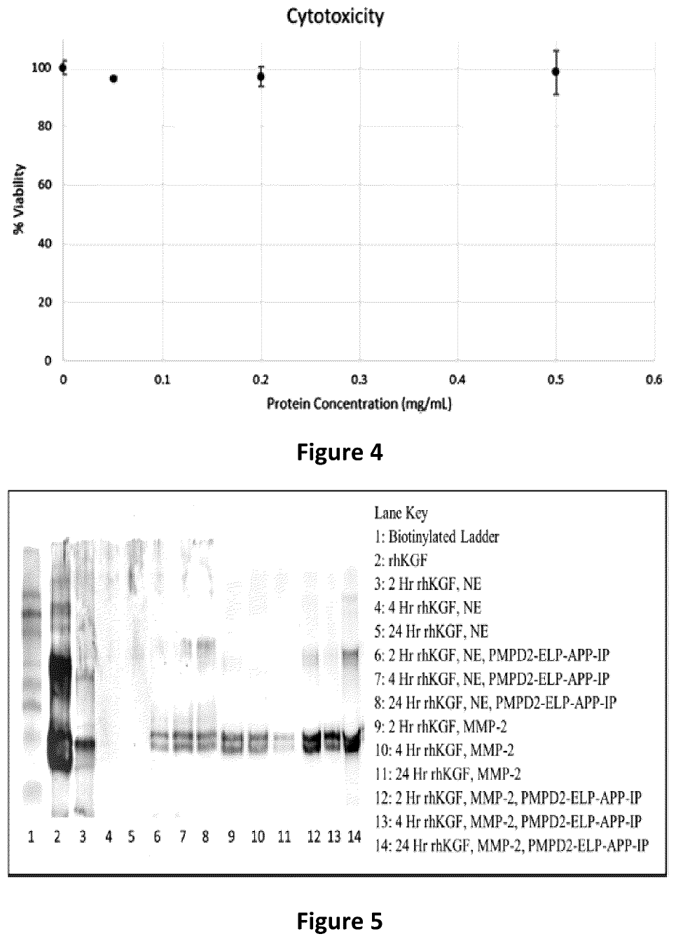

The dual protease inhibitor was incubated with cells to evaluate its possible toxic effects. There was no apparent toxicity at a range of concentrations of the dual protease inhibitor (FIG. 4). For concentrations as high as 0.5 mg/ml, no toxicity of the protease inhibitor was observed. This confirms that the inhibitor is nontoxic at its active concentrations.

Protection from Proteolytic Degradation of rhKGF

Recently, it was described that heterogeneous ELP based nanoparticles protect growth factors from degradation mediated by neutrophil elastase.¹² However, chronic wounds also have high MMP in addition to HNE. Thus, nanoparticles were assembled containing the growth factor (Keratinocyte Growth Factor, KGF) and the dual protease inhibitor. The NPs were incubated in both HNE and MMP-2 to evaluate protection of the growth factor in the NPs from degradation mediated by both proteases. It was observed that HNE degraded KGF completely in as soon as 4 h (FIG. 5, lanes 3-5). However, nanoparticles containing the dual protease inhibitor protected the KGF from degradation (FIG. 5, lanes 6-8). Similarly, MMP2 degraded the growth factor by 24 h (FIG. 5, lanes 9-11) but the NPs protected the growth factor from degradation (FIG. 5, lanes 12-14). The results confirm that the dual protease inhibitor protects the growth factor from both proteases thus eliminating the need of using two different protease inhibitors targeting each of the protease.

### Materials and Methods

### Plasmid Construction

The Elastin-like Peptide (ELP) used as a part of the fusion protein is L10FLAG, which is denoted by the sequence²⁷ [(VPGVG)₂(VPGLG)(VPGVG)₂]₁₀ DYKDDDDK (SEQ ID NO: 2). PMP-D2 is denoted by the amino acid sequence EEKCTPGQVKQQDCNTCTCTPTGVWGCTLMGCQPA (SEQ ID NO: 3). APP-IP is denoted by the amino sequence ISYGNDALMP (SEQ ID NO: 4). The nucleic acid sequences for the three amino acid sequences used to create PMPD2 L10FLAG APP-IP were obtained in plasmid form in pUC19 or pUC57 from GenScript®. The gene for PMPD2-L10FLAG-APP-IP was constructed by cutting PMPD2 from a pUC57 plasmid using PflMI and BglI restriction enzymes.₁₉ L10FLAG pUC19 was linearized using PflMI restriction enzyme.¹⁹ A ligation was performed between the PMP-D2 fragment and the linearized L10FLAG pUC19 to form PMPD2-L10FLAG pUC19 plasmid. Then PMPD2-L10FLAG was cut out of the pUC19 plasmid using PflMI and BglI restriction enzymes,¹⁹ while APP-IP pUC57 plasmid was linearized using PflMI. A ligation was performed between the PMPD2L10FLAG fragment and the linearized APP-IP pUC57 to form PMPD2-L10FLAG-APP-IP pUC57 plasmid. Using the same techniques PMPD2-L10FLAG and APP-IPL10FLAG was created to use as comparable inhibitors of NE and MMP-2, respectively.

### Protein Expression and Purification

The gene was cut out of the pUC57 plasmid using PflMI and BglI and each inserted into a pET25b plasmid. After confirmation of the correct genes using sanger sequencing, the plasmid was transformed into BLR(DE3) cells for expression of the protein.^{19,20} After a transformation one colony of the new plasmid was used to inoculate 75 ml of Terrific Broth growth media. After an 18-h incubation, the 75 ml culture was each placed into a liter of Terrific Broth growth media.¹⁹ After a 24-h incubation, the culture was pelleted and resuspended in 160 ml of 1×PBS. After resuspension, the sample was sonicated to lyse the cells and release the proteins into solution.¹⁹ Inverse phase transitioning was utilized to purify the protein from the cell lysate. For PMPD2-L10FLAG-APP-IP, its isoelectric point was utilized to purify the protein sample because of the fusion of charged regions on each side of the ELP. The cell lysate first underwent a centrifugation at 4° C. to remove cellular debris. Then the supernatant containing the proteins was collected and heated^{19,20} to 42° C. to transition the target protein, then centrifuged at the same temperature. After the hot spin cycle, the pellet was resuspended in 100 ml of cold 1×PBS. Three more cold and hot spin cycles were performed.¹⁹ The suspension of PMPD2-L10FLAG-APP-IP underwent a pH adjustment to the protein's isoelectric point before each hot spin cycle. After the cycles were complete, each sample was resuspended in 4° C. distilled water and dialyzed. After 24 h of dialysis, the samples were frozen and then lyophilized.

### Protease Inhibition Assays

Enzo Life Science's® colorimetric drug discovery kits and protocols were used for the evaluation of MMP-2 and NE inhibition for PMPD2-L10FLAG-APP-IP. Various concentrations of protein were used evaluated to locate the 50% inhibitory concentrations. The experiments were carried out in triplicate. Levels of inhibition for various concentrations of PMPD2-L10FLAG-APP-IP were compared to a control and the level of inhibition of experimental groups for both singular fusions, PMPD2-L10FLAG and APP-IP-L10FLAG to ensure dual fusion does not affect the inhibitory capabilities of the combinatory protein.

The NE inhibition assay was carried out by diluting the NE enzyme (2.0 mU/μl) 1/90 in NE assay buffer (100 mM HEPES, pH 7.25, 500 mM NaCl, 0.05% Tween-20), and 5 μl of the dilute NE was placed in each well of the 96-well plate for a final concentration of 0.22 mU per well (1 U=U=1.0 μmol of substrate per minute). The substrate (20 mM) was diluted 1/10 in assay buffer and 10 μl of the dilute substrate was placed in each well for a final concentration of 100 μM in each well. Appropriate amounts of assay buffer were placed in each well of the 96-well plate to bring the total volume up to 100 μl. Experimental groups were formed by using adding appropriated amounts of each inhibitor diluted in assay buffer to wells containing enzyme and substrate and the total volume was brought up to 100 μl for each well by adding assay buffer. For NE inhibition, the various concentrations of protein inhibitors tested were 5×10⁻⁷, 5×10⁻⁶, 1×10⁻⁵, and 5×10⁻⁴ μg/μl. A control (NE enzyme and substrate, no inhibitor) was used in triplicate. In addition, an in triplicate blank consisting of 90 μl assay buffer and 10 μl of dilute substrate was per well was used to blank the control. Protein blanks were also used in triplicate to ensure that the various concentrations of protein do not affect the measurement of absorbance. The average of the blanks was subtracted from the average absorbance of the control and similarly average absorbance of the blanks for each protein concentration was subtracted from average absorbance of the correlating protein concentration of the experimental groups. Table 1 represents the components of the control, blanks, and experimental groups. Before the substrate was added, the plate was warmed to 37° C. Then absorbance at 412 nm was measured over a 15-min period in 1-min intervals.

TABLE 1
Experimental design for the evaluation of NE inhibition

| Group | Contents |
|---|---|
| Blank | Assay buffer |
| Control | Assay buffer + NE + substrate |
| PMPD2-L10FLAG- | NE + substrate + protein at various |
| APP-IP (Exp. Group) | concentrations |
| PMPD2-L10FLAG | NE + substrate + protein in assay buffer at |
| (Exp. Group) | various concentrations |
| APP-IP-L10FLAG | NE + substrate + protein in assay buffer at |
| (Exp. Group) | various concentrations |
| Blanks for protein | Protein in assay buffer at various concentrations |
| exp. groups | |

Likewise, the MMP-2 inhibition assay was carried out similarly. MMP-2 enzyme was diluted 1/56 in MMP-2 assay buffer (50 mM HEPES, 10 mM CaCl2, 0.05% Brij-35, 1 mM DTNB, pH 7.5). Then 20 μl of the dilute enzyme was placed in each needed well of the 96-well plate for a final concentration of 1.6 U per well (1 U=100 pmol of substrate per minute). The MMP-2 substrate (25 mM) was diluted 1/25 in MMP-2 assay buffer and 10 μl of diluted substrate was added to each needed well of a 96-well plate for a final concentration of 100 μM in each well. Appropriate amounts of assay buffer were placed in each well of the 96-well plate to bring the total volume up to 100 μl. Experimental groups were formed by adding appropriate amounts of each inhibitor diluted in assay buffer to wells containing enzyme and substrate and the total volume was brought up to 100 μl for each well by adding assay buffer. The various concentrations of each protein inhibitor tested for MMP-2 inhibition was 0.004, 0.008, 0.01, 0.05, 0.2, and 0.5 μg/μl. A control (MMP-2 enzyme and substrate, no inhibitor) was used in triplicate. In addition, an in triplicate blank consisting of 90 μl of assay buffer per well and 10 μl of dilute substrate was used to blank the control. Protein blanks were also used in triplicate to ensure that the various concentrations of protein do not affect the measurement of absorbance. The average of the blanks was subtracted from the average absorbance of the control and similarly average absorbance of the blanks for each protein concentration was subtracted from average absorbance of the correlating protein concentration of the experimental groups. Table 2 represents the components of the control, blanks, and experimental groups. Before the substrate was added, the plate was warmed to 37° C. Then absorbance at 412 nm was measured over a 15-min period in 1-min intervals. The average absorbance of each kinetic reading for the control and experimental groups were blanked. Data analysis was accomplished by normalizing the slopes of the blanked experimental groups at various concentrations and time points to the control to evaluate percent inhibition.

TABLE 2
Experimental design for the evaluation of MMP-2 inhibition

| Group | Contents |
|---|---|
| Blank | Assay buffer |
| Control | Assay buffer + MMP-2 + substrate |
| PMPD2-L10FLAG- | MMP-2 + substrate + protein in assay buffer at |
| APP-IP (Exp. Group) | various concentrations |
| PMPD2-L10FLAG | MMP-2 + substrate + protein in assay buffer at |
| (Exp. Group) | various concentrations |
| APP-IP-L10FLAG | Assay buffer + MMP-2 + substrate + protein at |
| (Exp. Group) | various concentrations |
| Blanks for protein | Protein in assay buffer at various |
| exp. groups | concentrations |

### Cell Toxicity Experiment

A549 cells were plated in a 24-well plate at a density of 50,000 cells per well in DMEM 10% FBS 1% AA. After 24 h, the media was removed from the wells and replaced with serum free DMEM 1% AA with three different concentrations of protein and a control absent of protein in triplicate. After 24-h incubation with the protein, a Hoechst assay was performed by removing the media from each well on the 24-well plate. Then the cells were washed using 1×PBS. After being washed the 400 μl of distilled H2O was added to each well and a freeze thaw cycle was performed by placing the 24-well plate in −80° C. for 10 min and then thawed in 37° C. for 30 min. The freeze thaw cycle was performed three times to ensure the cells were lysed. Next 100 μl of Hoechst solution with 100 μl of each sample was added to a 96-well plate. The plate was shaken for 30 s and fluorescence was read in a plate reader at 360 nm excitation and 460 nm emission. A triplicate blank of consisting of Hoechst solution was subtracted from the control and the experimental groups.

Protection from Proteolytic Degradation of Growth Factor, rhKGF

PMPD2-L10FLAG-APP-IP was used to evaluate the protection of KGF from degradation due to NE and MMP-2. A recombinant KGF was used by creating a 10 μg/ml stock solution of rhKGF. From the stock solution 2.5 μl of 10 μg/ml rhKGF was mixed with 5 μl of a 10 mg/ml stock solution of PMP-D2L10FLAGAPP-IP in NE assay buffer. HNE (2.0 mU/μl) obtained from Enzo Life Sciences®. Then 1.56 μl of the dilute NE was added with 15.94 μl of NE assay buffer to bring the total volume up to 25 μl with a final HNE concentration of 250 ρU/μl.¹² A positive control was made by mixing 2.5 μl of 10 μg/ml rhKGF with 1.56 μl of NE and 20.94 μl of NE assay buffer. An original sample was made by mixing 2.5 μl of 10 μg/ml rhKGF and 22.5 μl of NE assay buffer. The same was done for MMP-2. MMP-2 (150 U) from Enzo Life Sciences® was obtained and reconstituted. The final experiment concentration of MMP-2 was 11.6 mU/μl by mixing 3 μl of MMP-2 stock solution with 2.5 μl of rhKGF 10 μg/ml stock solution and 5 μl of 10 mg/ml PMP-D2-L10FLAG-APP-IP and then brought up to a 25 μl mixture by adding MMP-2 assay buffer. A positive control was also made by mixing 3 μl of MMP-2, 2.5 μl of 10 μg/ml rhKGF, and assay buffer to make a 25-μl mixture. The five microcentrifuge vials with the original sample, controls, and the experimental groups were placed in a 37° C. hot bath and incubated for 24-h. Throughout the incubation period, 6 μl samples were taken from the positive controls and the experimental groups at 2, 4, and 24 h.¹² After 24 h, a 6 μl sample was taken from the original sample to evaluate degradation in the positive control and the experimental group. The 6 μl samples were annealed at 96° C. with 1×DDT/Red Loading Dye and then they were placed in an SDS-PAGE gel with an 8 μl of biotinylated ladder and ran for 1 h at 150 V in 1×Running Buffer. After running for 1 h, the samples were transferred to a membrane in 1×Transfer Buffer for 1 h at 350 mA. A TBS/Tween (100 ml of 10×TBS+900 ml of diH2O+500 μl of Tween) solution was made. After the 1 h transferring procedure, membrane blocking was done by making a 10 ml 5% milk/TBS/Tween solution and agitated with the membrane for 1 h. After 1 h, the 5% Milk/TBS/Tween solution removed and replaced with a 5% Milk TBS/Tween solution with 14 μl of Rabbit Anti-Human KGF antibody and incubated over night while agitated at 4° C. After overnight incubation, the membrane was washed three times for 5-min intervals in 10 ml of TBS/Tween. Then the membrane was agitated for 1 h in 5 ml of 5% Milk TBS/TWEEN solution containing 2 μl of Anti-Rabbit antibody and 10 μl of Anti-biotin. After the 1-h incubation, the membrane was washed three times in TBS/Tween solution for 5-min intervals. After three washes, the membrane was placed in 10 μl of exposing solution (9 ml of diH2O+500 μl of lumiglo+500 μl of peroxide reagent) and agitated for 1 min. The membrane was then exposed to chemiluminescence for 15 min and an image was taken.

### Statistical Analysis

For each kinetic inhibition experiment for both NE and MMP-2, each protein and protein concentration were tested in triplicate (N=3) and standard error of the mean absorbance for each data point was calculated. The slope of latter inhibition curves for each protein at each concentration was normalized to the control's slope. From the latter, percent inhibition for each protein concentration was calculated.

Statistical analysis of the cytotoxicity experiment was performed by normalizing the average fluorescence of experimental groups to the average fluorescence of the control. Then standard deviation of the normalized data was calculated and applied to the graph displaying the results of the Hoechst assay.

### Example 2—Wound Healing System

The most basic understanding of the process involved in wound healing is the balance between degradation of damaged tissue and deposition of a new healthy tissue with a strong Extracellular Matrix (ECM). Wound healing can be delayed if the immune system's process of breaking down the tissue overpower deposition over longer periods than normal.

Regenerative approaches have been used to try healing damaged tissues, but are commonly treatments that include and require live, viable, stem cells or solubilized proteins require unique and expensive storage conditions (−80° C. and liquid nitrogen) and are therefore limited in shelf life, shipping constraints, and application styles.

The inventors propose that the important aspect to stem cell based regenerative therapies is not necessarily delivering stem cells to damaged tissue, where the stem cells most likely quickly die, but rather the many proteins they create that aid in wound healing processes. Immediately, current chronic wounds would benefit from a therapeutic approach that simply focuses on closing a wound through rebalancing tissue degradation and deposition, while focusing on preventing new chronic wound formation on a patient through the advocacy of lifestyle changes and an immediate pursuit of medical treatment when a patient with chronic wound history obtains a new wound.

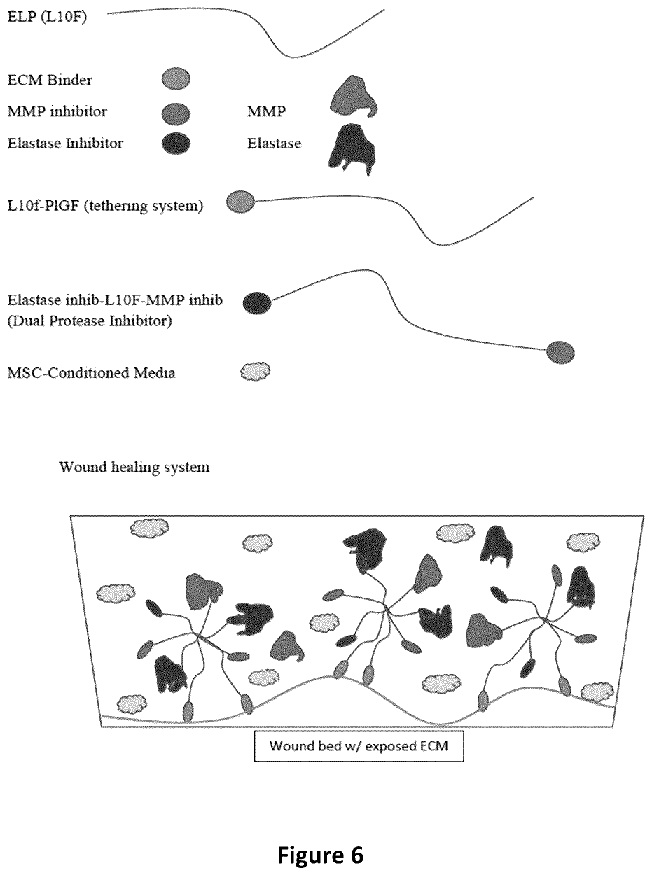

A wound healing strategy to target chronic and hard-to-heal wounds (HtHW) was formulated to interrupt the system of balance of tissue destruction and deposition, while keeping in mind the economic impacts and inherent burdens the current treatment of chronic and HtHWs incur. A protein-based therapeutic agent was designed as a three-component system that includes (1) a multifunctional protease inhibitor, (2) a conditioned media for inducing cellular proliferation, and (3) a tethering protein comprised of an ECM targeting peptide to target and extend delivery of the protease inhibitors around essential tissue structures that are susceptible to proteolytic degradation. The wound healing system is generally shown in FIG. 6.

The composition is generally a dry acellular mixture that can be applied dry, reconstituted, or embedded in materials to cause closure of HtHW, normal wounds, and chronic wounds. In one embodiment, the composition may be a freeze-dried, powder-based product, with an extended shelf-life, and ease of application of a powdered product that for example is less affected by factors such as gravity on a flowing liquid. The benefit of a dry product is that it can be stored in ambient conditions and solubilized for delivery, applied dry directly in a wound, mounted in a gel for sustained release, embedded in materials, and even applied as a powder coating on a bulk material.

While the compositions and method are described as pertaining to wound healing and tissue damage, they are not limited to such. Given that the composition can be produced as a dry mixture, as well as the known immunomodulatory effects of MSC's, the composition can easily be incorporated into material manufacturing of implant devices to increase the healing time of damaged tissue and decrease the immune system's rejection of the device.

The three main components of the composition act together to provide several different functionalities while traditional approaches typically only have one useful application per therapeutic. These functionalities include, but are not limited to: a plurality of specific growth factors for growth; blocking of multiple proteases; sustained release via a molecular tether; sustained release due to aggregation properties of ELPs; simplification of purification steps for recombinant protein additives; and the dry nature of the composition allows for easy/low cost storage and application of the product in less than desirable environments (war zones, pasture grazing animals, etc.). It is also advantageous that the “recombinant protein” additives can be constructed from mammalian, including human, sequences which allows for easier analysis of toxicity and side effects which may occur when introducing proteins with entirely novel sequences that are not seen naturally in the body. This allows for easy alteration of the current described treatment to match exactly the issues seen in a specific wound condition. Each of the components of the novel composition are discussed in more detail below.

### Conditioned Media

Human Mesenchymal Stem Cells (MSCs) have been widely used in numerous ways to treat a variety of diseases. Most notably, mesenchymal stem cells harvested from the umbilical cord, bone, amniotic fluid, and adipose tissue⁴⁰ have commonly been used as a cellular-based therapy for the treatment of diseases that cause tissue degeneration or debilitating tissue damage. Their attractiveness for cellular therapy is wide spread, where they are utilized as a whole cell for their multi- and pluripotency that in theory lend them useful in application in the body to seed new cells of the damaged tissue type⁴⁰⁻⁴² Furthermore, their application has gone as far as to suggest a usefulness of the products contained inside of extracellular vesicles produced and secreted by MSC's⁴². With many application styles of MSCs and their products they produce, the commonality in their use is the pursuit of promoting tissue healing. Whether that is through the delivery of the pluripotent cell with the capacity for differentiation⁴⁰⁻⁴², the immunomodulatory and anti-inflammatory qualities of MSCs⁴⁰, or for the various regenerative biproducts that MSCs produce to influence cellular migration, growth, and protein deposition for complete tissue repair⁴³.

Regarding cellular biproducts created by MSCs, this has been previously studied to some degree where it was determined that conditioning biproducts of MSCs include some very prominent wound healing peptides such as EGF, TGFa, TGFb1, VEGF, FGF2, as well as many immunosuppressing and modulating proteins⁴³. Wound healing and tissue regeneration rely on a lot of these growth factors and peptides in natural tissue healing processes. Vascular endothelial growth factor (VEGF) is important to stimulate remodeling processes like cellular migration and proliferation³⁸. Endothelial growth factor (EGF) is important for stimulating re-epithelialization of a wound, while transforming growth factors such as TGFb1 and TGFa aid in re-epithelialization but also in matrix formation and remodeling of the wound³⁹ which is important for regaining structural integrity in newly healed or formed tissue. Similarly, Fibroblast Growth Factor 2 (FGF 2) helps aid in the remodeling and matrix formation process³⁹. Growth factors such as EGF, TGFa, TGFb1, FGF2, and VEGF have all been found to increase in levels in a naturally healing acute wound with no therapeutic intervention, but have been seen in decreased levels in chronic wounds³⁹.

Due to current knowledge of chronic wounds and that there are growth factor species whose levels can be correlated with an acute healing wound versus a chronic wound¹⁷, it is advantageous to replace the growth factors that are seen in suppressed levels in chronic wounds to move the wound to closure. As previously drawn, there is a correlation, between the proteins needed in wound healing, and those that have been found to be produced by mesenchymal stem cells. Therefore, a great candidate for proliferation inducing therapies to aid in the remodeling of hard-to-heal or chronic wounds, is media conditioned by stem cells, which include proteins that are essential to cellular migration, proliferation, and remodeling of the wounded tissue. A protein cocktail, from the conditioning of media, by mesenchymal stem cells can easily be mixed with other protein-based therapeutics, such as the multifunctional protease inhibitor described herein to create a self-protecting therapeutic to aid in the downregulation of protein destroying proteases found in high levels in chronic wounds, while delivering and protecting present growth factors necessary for tissue regeneration of the skin.

The conditioned media acts as a proliferation inducing component and contains wound healing peptides and proteins secreted by umbilical cord derived mesenchymal stem cells as well as numerous growth factors commonly observed in the body during normal wound closure. In some embodiments, the conditioned media may be a blend of freeze-dried peptides that can be applied to a wound to increase proliferation and remodeling. In contrast, to create a similar cocktail of proteins recombinantly or synthetically, it would require separate processes for each desired protein. The overall product solves problems in reference to protein synthesis and purification as MSC conditioned supernatant contains a variety of growth factors (not just one, like recombinant approaches where more proteins require more reactors and more purification steps are needed).

The media conditioning process involves a staggered collection approach, where only a partial volume of the conditioned media is removed from the cell culture dish and replaced with fresh media. Utilizing this approach allows for a more potent conditioned media, as well as takes advantage of possible cellular signaling and feedback mechanisms that puts the cells in culture into a state of senescence that can be maintained long term, while still producing conditioned products, without spending cellular metabolic processes and nutrients on cellular division.

After the conditioned media is collected it then undergoes dialysis to remove any unwanted salts and small molecules. With relation to the MSC conditioned media, each product can be tested upon production for proliferative effects to verify efficacy. Using the novel protocols previously created by the inventors, a dish of cells can be maintained for weeks to months without further proliferation while still producing a potent conditioned media. Once dialysis is complete the product is then freeze dried into a resulting powder which can then easily be mixed, dry, with the engineered proteins, which are expressed in bacterial and then purified using inverse transition cycling (ITC). Once purified the protein product is freeze dried into a resulting powder.

Umbilical cord MSCs have been known to be immunomodulatory in a fashion where their cytokines elicit a minimal immune response if any at all. This is key for the aspect of utilizing the dry product embedded in implantable materials to not only induce cellular proliferation in the implant area but also minimize rejection of the implanted material by masking it with the conditioned media products that do not elicit an immune response.

Induction of Proliferation of C2C12 Cells Treated with Um-MSC Conditioned Media with or without the Presence of Proteases

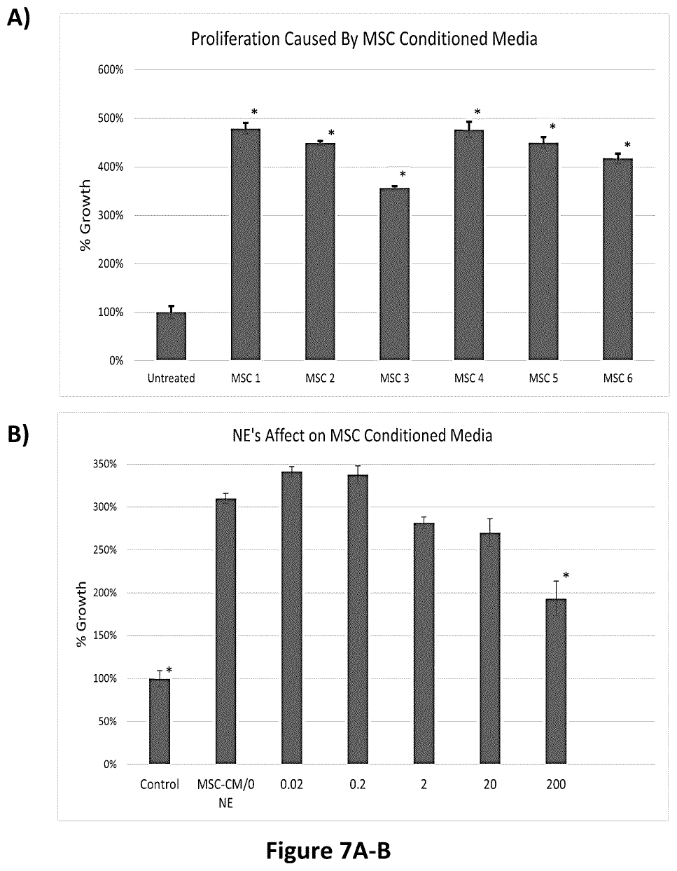

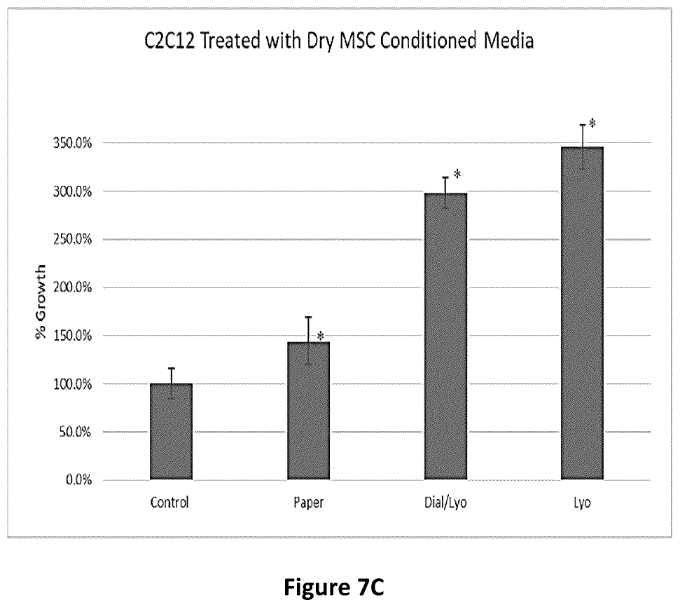

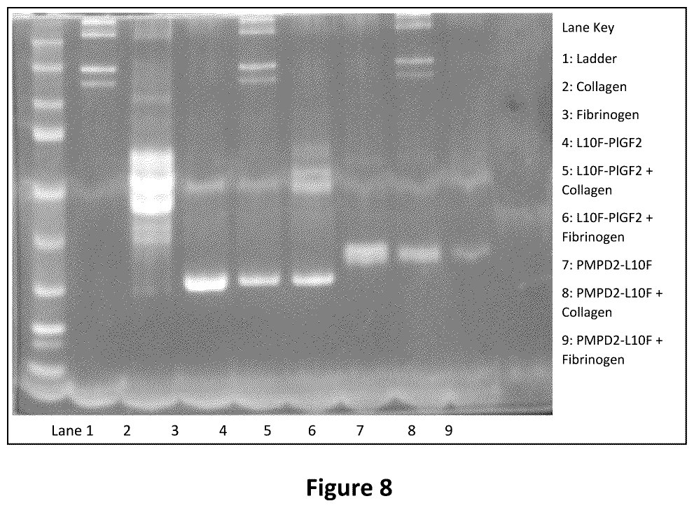

Umbilical cord derived MSC conditioned media obtained from the harvesting of the used media from MSC's described previously was used to induce proliferation in C2C12 mouse myogenic cells in vitro. FIG. 7A displays the experimentation with various samples from different collections of um-MSC conditioned media. Here, it is observed that samples such as MSC 1 and MSC 4 induced a 4.8-fold increase in cellular proliferation when compared to the untreated control. MSC 3 in FIG. 7A, caused the least amount of proliferation, but was still 3.6-fold higher proliferation than that of the untreated control.

FIG. 7B displays the results of a proliferation assay with C2C12 cells treated with conditioned media, and various concentrations of NE. Um-MSC conditioned media sample MSC 3 from the experiment results reported in FIG. 7A was the sample used for treatment with the various concentrations of NE and showed similar induction of proliferation, in FIG. 7B when NE-0 mU/mL. This is important to note as it expresses the consistency in capturing proliferation induction using this assay. Though, in FIG. 7B when analyzing the effect of NE on conditioned media treatments, it is seen that at 200 mU/mL of NE there is a significant decrease in C2C12 proliferation, but as the concentration of NE is lessened, proliferation steadily increases back to the degree of proliferation observed in the MSC conditioned media treatment with no NE. In FIG. 7B, there is a 1.6, 1.14, and 1.1-fold decrease in proliferation, for 200, 20 and 2 mU/mL concentrations of NE respectively, compared to cells that were treated with just MSC conditioned media. At an NE concentration of 0.2 and 0.02 mU/mL it is observed that proliferation is that of the C2C12 cells that were treated with just MSC conditioned media, no inhibitor. The latter result drives the importance in combining the dual protease inhibitor with um-MSC conditioned media.

FIG. 7C displays the proliferation results when utilizing MSC conditioned media in dry forms in vitro. As it is observed in FIG. 7C, all dry processed um-MSC conditioned media caused proliferation over that of the untreated control. The MSC conditioned media embedded filter paper had a 1.44-fold increase in proliferation over the degree of proliferation of untreated control. While lyophilized products that were dialyzed, or not, and used in powder form showed a 2.98 and 3.50-fold increase in proliferation. These results align closely with liquid-based treatments, except for the filter paper embedding of MSC conditioned powder and the deviation here is due to the fact that there is volume limitations for embedding the powder within a thin piece of material.

### Multifunctional Recombinant Protease Inhibitor

Wounds, including chronic wounds, traditionally see levels of proteases secreted by immune cells in response to wounding for the purpose of tissue breakdown. Though tissue degradation is a necessary part of wound healing, wounds can be healed faster by inhibiting proteases that not only degrade the necessary structural tissue components, but also degrade growth factors that are present and necessary for tissue construction. Therefore, the composition described herein employs a fusion protein that acts as a multifunctional protease inhibitor, as more fully described in Example 1 above. Example 1 describes an exemplary engineered protein comprised of two inhibitors known to block elastases and MMPs (dual protease inhibitor). This multifunctional protease inhibitor was added to the final dry acellular conditioned media product to protect the growth factors gained from the MSC media conditioning as well as the healthy tissue and structural components.

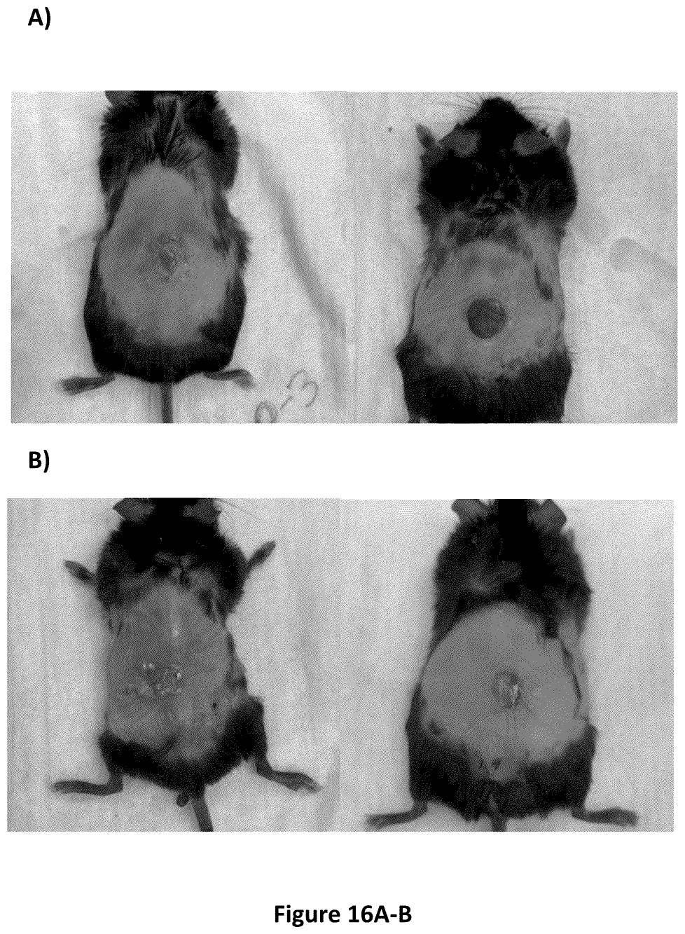

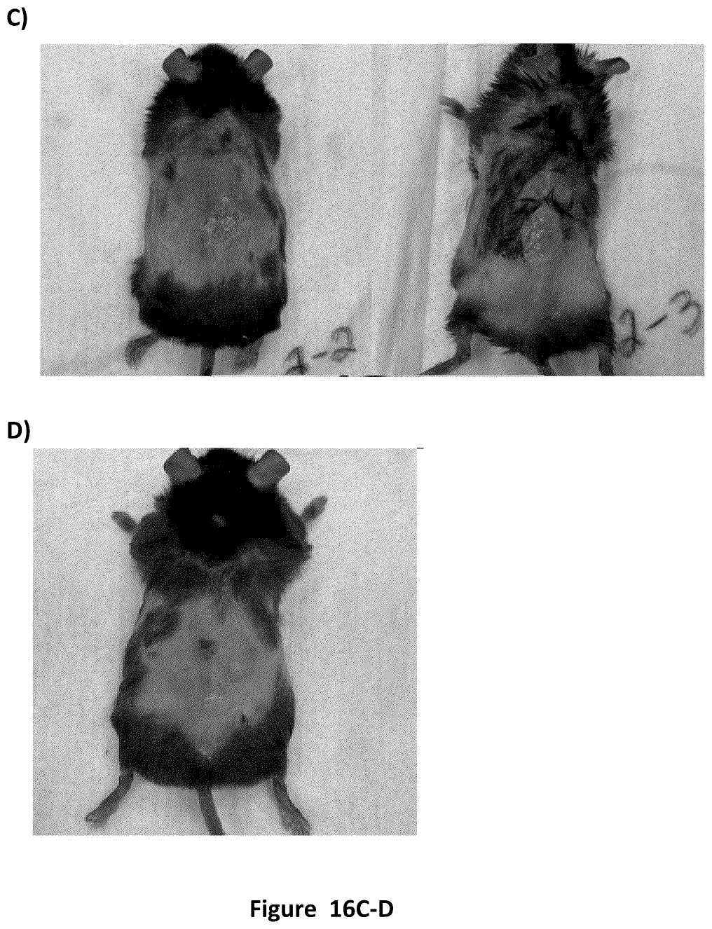

This dual protease inhibitor has been shown to be effective in vitro (see Example 1) as well as in vivo (diabetic mice as described later herein). (see FIGS. 16-19). The conditioned media products exposed to proteases are less effective in inducing proliferation compared to conditioned media products not exposed to proteases or conditioned media products supplemented with a dual protease inhibitor while exposed to proteases.

The dual protease inhibitor is a single protein with multiple inhibition targets where the biologically active domains are centered around an elastin-like polypeptide (ELP) for spacing purposes. The ELP spacer is essential to maintain biological activities of the protein so it can act simultaneously against multiple proteases. If the spacer was too short the protein would lack the ability to inhibit both MMPs and elastase at the same time.

Utilizing an ELP as a spacer allows a multifunctional protein to be constructed and maintain specific activities simultaneously. This allows for protein production of a single protein rather than having to produce two entirely different proteins separately, in separate reactors, and then additional mixing of therapeutics be required. The latter lowers cost/time to produce the inhibitor product while maintaining all activities.

The recombinant protein is not limited to two biologically active domain fusions. A protein such as Bioactive molecule-ELP-Bioactive molecule-ELP-Bioactive molecule, can be constructed to gain a protein with 3 inhibitory functions. Similarly, additional different bioactive molecules may be added with ELP spacers positioned between. Recombinant proteins constructed with 3 or more inhibitory functions can be advantageous as additional bioactive domains can target other proteases such as bacterial proteases that break down human tissue in infected chronic wounds.

### Tethering Peptide

The feature of using ELP fusion proteins for sustained presence in the application site is inherently lost for fusion proteins with a high degree of hindrance of self-assembly due to charge. To implement a fail-safe method of sustaining fusion protein presence in the application site and around ECM components that require protection from proteolytic degradation, the inventors constructed other ELP fusion proteins to include a bioactive domain with Extracellular Matrix affinity. For example, an additional recombinant protein, a tethering protein, was developed which is comprised of a biologically active peptide sequence of placental growth factor 2 (PlGF2) fused to a free end of an elastin-like peptide, L10f, to create L10f-PlGf-2. The biologically active region of PlGF2 is known to bind to extracellular matrix components such as collagen, elastin, fibrin, fibronectin, fibrinogen, etc.³⁵ In the exemplary composition described herein, this tethering peptide performs the duty of tethering PMPD2-L10Flag-APPIP. The free ELP end inherently interacts with the ELP portion of PMPD2-L10Flag-APPIP. While the PlGF2 end interacts and binds to extracellular matrix components essentially tethering the dual inhibitor to the wound bed. An experiment was ran where 1 mg/mL samples of L10Flag-PlGF2 were incubated at 42° C. for 10 minutes, to transition with collagen, fibrinogen, or alone. Then each sample was centrifuged at 15,000 rpm for 5 minutes. The supernatant was discarded and the resulting pellet was resuspended in 500 μl of 1×PBS. A 6 μl sample was taken from each resulting suspension, and a total protein stain was performed on an SDS-Page gel and stained with simply safe blue (Coomassie G250), and gel pictures were taken and analyzed.

The inventors have shown that L10f-PlGf-2 binds to ECM components. FIG. 8 displays a total protein staining result of the interaction of PlGF2 with fibrinogen and collagen. Here it is observed, in lanes 3-5, that L10Flag-PlGF2 has affinity for, and under centrifugation, pulls fibrinogen and collagen out of solution when heated and self-assembled. When compared to a single fusion of PMPD2-L10Flag in lanes 7-9, it is observed that L10Flag or PMPD2 may have some affinity for collagen already, but no affinity for fibrinogen. Whereas with L10Flag-PlGF2 affinity for fibrinogen is observed in lane 5.

The free L10 (ELP) portion of the recombinant protein is left free and unhindered to bind with other ELP regions on the multifunctional protease inhibitor to provide immediate protection for ECM components degraded by proteases. Additionally, this can be utilized for tethering antimicrobial ELP-fusion proteins to protect tissues from bacterial invasion and further proteolytic breakdown. This construct establishes a mechanism to provide immediate, local, and sustained protection of not only the MSC conditioned product, but also the ECM components that are essential for tissue reconstruction and cellular migration. Additionally, this tethering system can be employed with any fusion protein created with a ELP, such as L10flag, core.

Verification of pI-Based Phase Separation for the Purification and Application of ELP Fusion Proteins

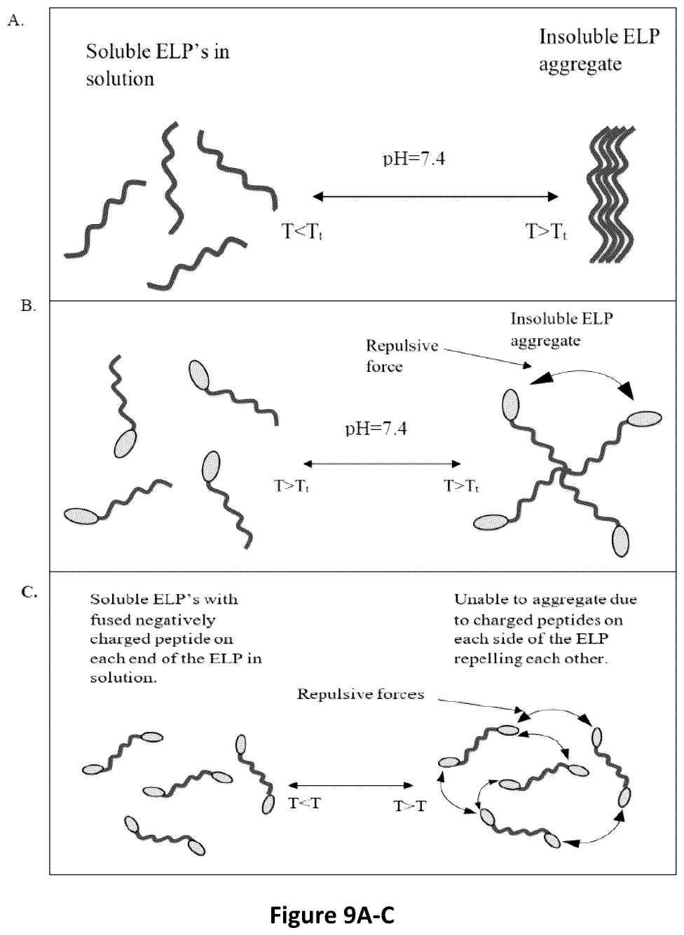

Unsuccessful purification of ELP-fusion proteins such as, PMPD2-L10Flag-APPIP, lead to a hypothesis that ELPs centered between two bioactive regions with similar charge polarities are hindered during temperature dependent aggregation and will not self-assemble to a degree necessary for bulk phase separation. FIG. 9 displays the ELP self-assembling behavior at a neutral pH, for three different cases of fusions. FIG. 9A displays the case where there is a unfused ELP. An ELP without fusions is completely unhindered by charges because the ELP sequence is largely non-polar allowing for easy phase separation at a neutral pH. This can be seen in FIG. 9A where the ELPs readily aggregate at a temperature (T) above that of the ELPs' transitioning temperature (Tt). FIG. 9B presents a case of ELP fusion that contributes hinderance to the overall behavior of aggregation. This case is a single charged fusion at one end of the ELP. Though ELP transitioning can still occur, the degree of transitioning is controlled by the charge density of the fused domain on the end of the ELP. In FIG. 9B the aggregate formed is highly dependent on how close two identically charged ends, from two individual molecules, can get before repulsive charges limit ELP interactions at the core of the aggregate. Additionally, FIG. 9C shows another case where there is a charged domain fused to each end of the ELP creating a molecule with a non-polar ELP core and two ends of the molecule containing similar repulsive charges. In this case, at a neutral pH, little to no aggregation will be accomplished when solution T≥Tt because individual molecules and their ELPs will never get close enough to interact and self-assemble due to the repulsive forces between the ends of each bioactive domain during intermolecular interaction.

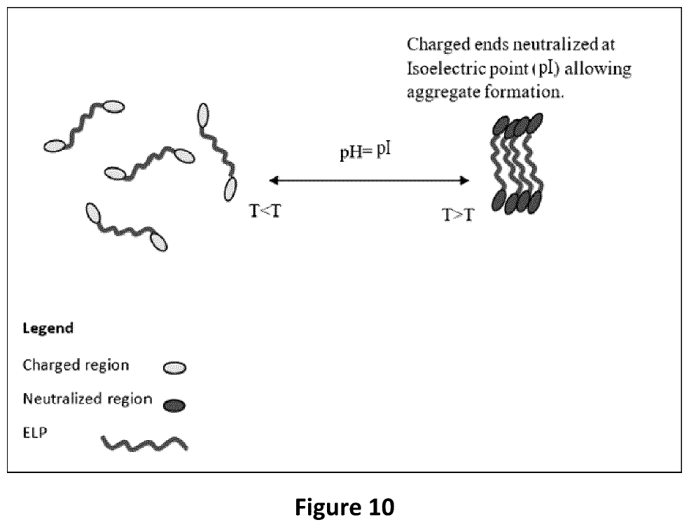

The reversable elimination of charges on the ends of ELP-fusion proteins allow for the self-assembly and phase transition necessary to utilize ELPs and their transitioning capabilities for purification and application. PH variations of a protein solution to the protein's isoelectric point can reversibly neutralize charges to allow for aggregation when deemed useful. For example, for protein purification, inverse transition cycling of ELP-fusion proteins can be utilized when highly charged bioactive ends exist through the employment of pH adjustments before hot centrifugations by adjusting the solution pH to the protein's isoelectric point (pI). This eliminates charges for as long as the pH is at the proteins pI. In FIG. 10, this hypothesis is observed, and suggests when a protein solution pH is adjusted to that of the protein's pI, ELP interactions are therefore enabled (when T≥Tt) through the elimination of repulsive charges that exist during an intermolecular interaction at a neutral pH in FIG. 9C.

Absorbance Results for pI-BPS of ELP Fusion Proteins

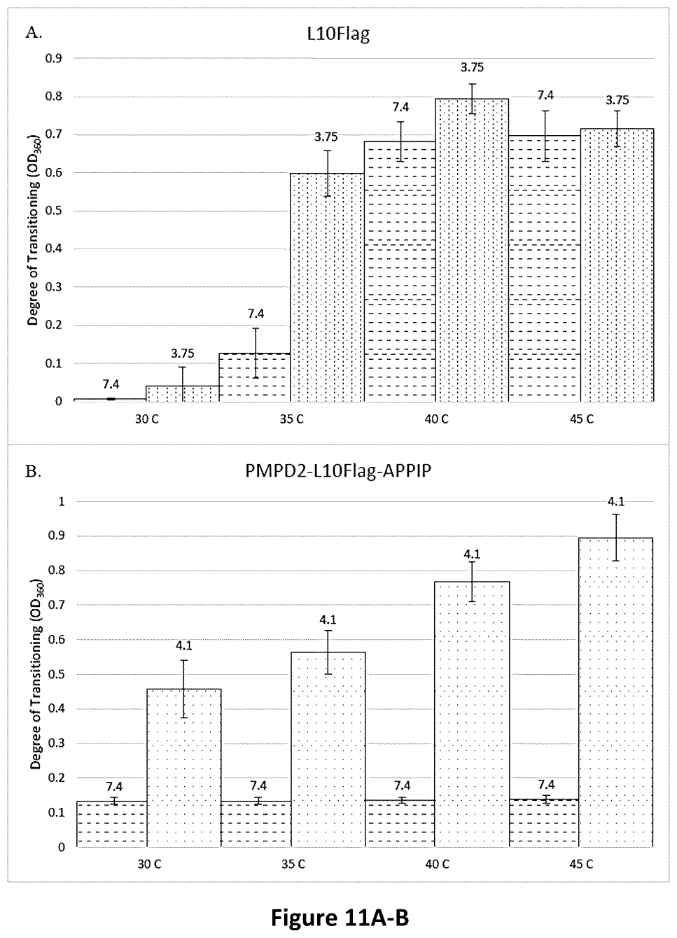

Absorbance measurements of transitioning for ELP fusion proteins confirmed the importance of pI-BPS for the purification and application of these fusion proteins. L10-Flag is the most basic ELP fusion protein that we work with and contains little hinderance to transition since any charge on this protein is from the fusion of a Flag-tag end. FIG. 11A represents a graph of L10 Flag and the degree of transitioning over a range of temperatures at 7.4 and 3.8 pH. Here, it is seen that L10Flag transitions to nearly the same degree at its pI and above its Tt the sample transitions like at a neutral pH. However, at or near L10Flag's transition temperature it is clear that the sample at its pI transitions nearly 5-fold better than at a neutral pH. FIG. 11B displays transitioning results of the dual protease inhibitor, PMPD2-L10Flag-APPIP. It is clear that pI-BPS is needed for the ITC purification of this protein. Over the range of tested temperatures, the protein sample at a neutral pH, did not have an elevated increase in absorbance due to transitioning. In fact, the level of transitioning stayed very similar over the tested temperature ranges. At PMPD2-L10Flag-APPIP's pI, 4.1 pH, the transition steadily increased over the range of temperatures reaching a 6-fold increase in absorbance than that of the sample in a neutral buffer.

Through a ITC purification process of PMPD2-L10Flag-APPIP, it was clear that pI-BPS implemented in the ITC protocol was absolutely necessary to maximize protein yield just by observation of end result. Purifying two batches of PMPD2-L10Flag-APPIP alongside one another, with one batch kept at a neutral pH throughout, and the other following pH adjustments for hot centrifugation spins at the proteins PI, further proved the necessity of pI-BPS. The yield in protein for the PMPD2-L10Flag-APPIP, at its pI, was a 22.5-fold increase over the protein yield of the sample that remained at a neutral pH throughout.

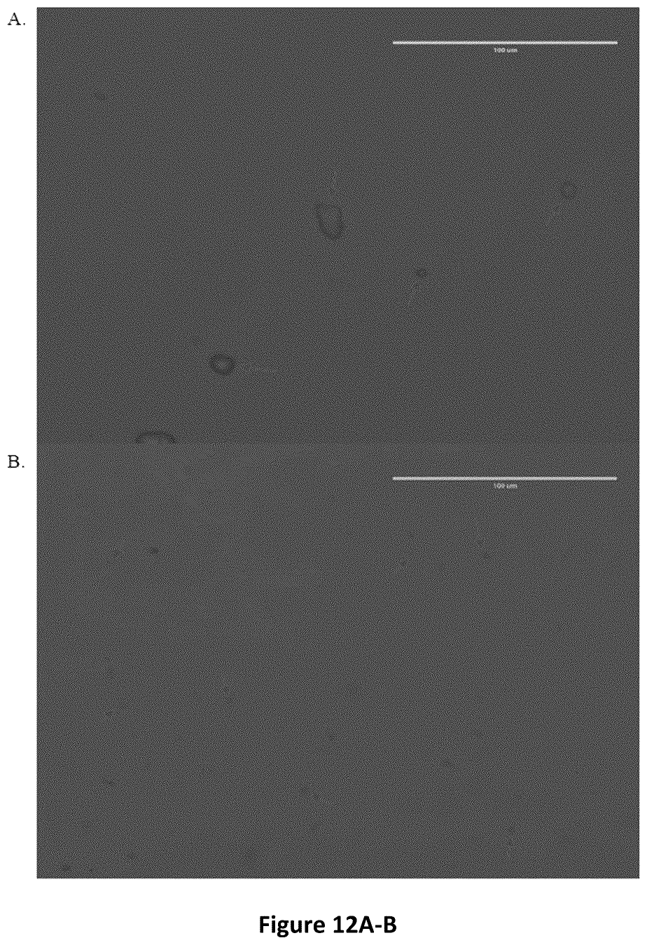

Samples of PMPD2-L10Flag-APPIP were further analyzed at the proteins pI and at 7.4 pH. Samples were transitioned and viewed under a microscope to observe size differences in self-assembled particles. FIGS. 12A and B show qualitatively the very large difference in particle size when each solution is heated. This observational result further shows the effect of pI-BPS on ELP-based fusion proteins and their transition dependency on solution pH (or protein charge).

Dynamic Light Scattering Analysis of Particle Size Formation Utilizing pI-BPS

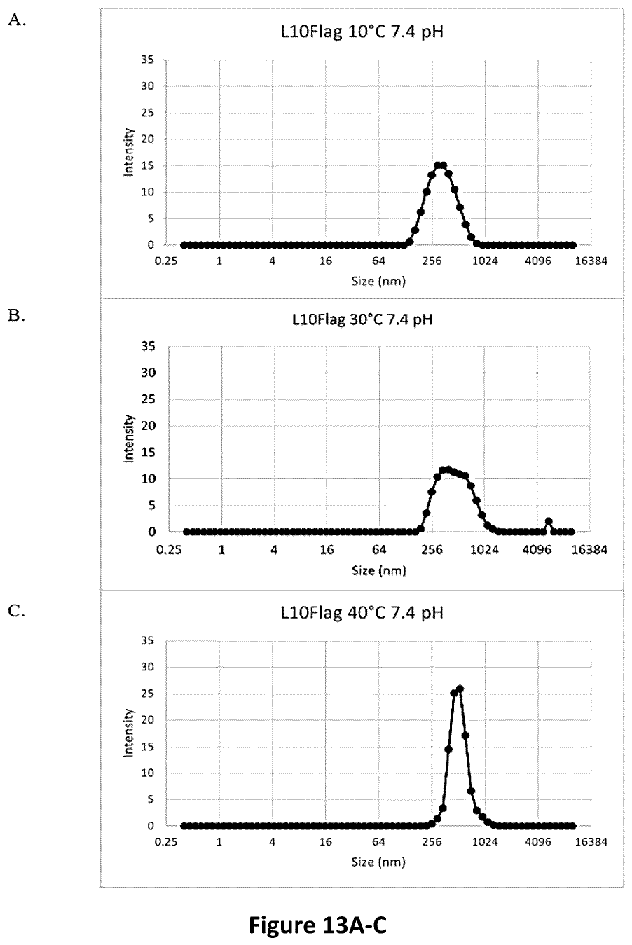

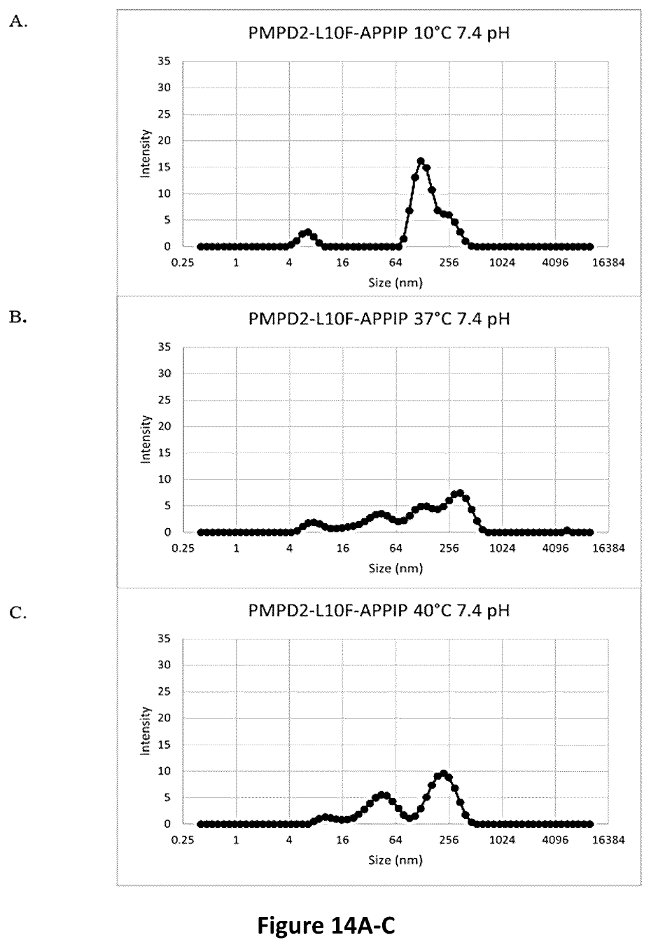

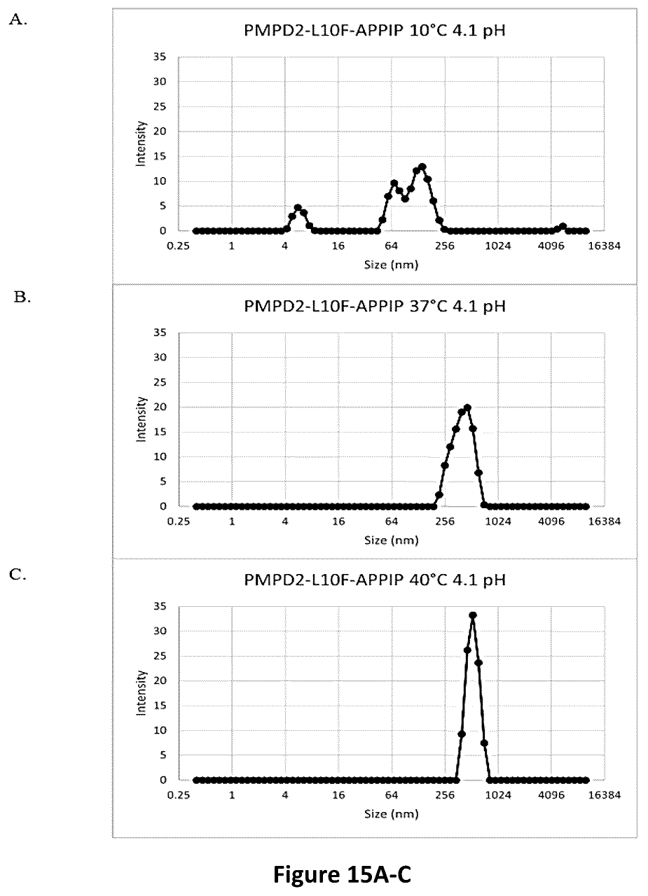

The DLS data further confirms what has been observed with microscopy, absorbance experiments for transitioning, and purification results. FIG. 13A-C represents the size distribution data collected on L10Flag from 10-40° C. FIG. 13A shows particle size data of a un-transitioned sample of L10Flag in a neutral buffer at 10° C., which displays a small particle size in low amount. FIG. 13B shows L10Flag's particle size distribution at 30° C., where it can be observed that a small shift to a larger particle size is taking place with transitioning beginning to occur. Transitioning is apparent in FIG. 13C displaying L10-Flag transitioning further at 40° C., which is indicated by a further peak shift, 1.6-fold higher than 10° C., and the intensity of this particle size being nearly 1.7-fold higher that of the un-transitioned protein in samples at 10° C. and 30° C. The progression of transitioning, with temperature rising at a neutral pH, can be seen quite clearly through peak shifts and elevated intensity in FIG. 13A-C. To show the effect of highly charged ELP-fusion proteins, FIG. 13A-C is DLS data for PMPD2-L10Flag-APPIP in a neutral buffer. Here in FIG. 14A it can be seen that PMPD2-L10F-APPIP particle sizes at a low temperature of 10° C., at their largest are 160 nm in size with a low intensity value of 15. When temperatures are increased, to 37° C. and then 40° C. in FIG. 14B-C many more peaks or species are observed, with very low intensity, suggesting transitioning is being hindered and ultimately will not occur when compared to the results seen in L10Flag in a neutral buffer. This observation alludes to the fact that transitioning is occurring, but hindered, and to a very small degree (particle sizes as low as 45 nm, FIG. 13C.) and in accordance with microscopy in FIG. 12B where smaller particles are observed. In contrast, PMPD2-L10Flag-APPIP in a buffer at its pI, displays a large degree of transitioning (like that of unhindered L10Flag), which can be seen in FIG. 15A-C when temperature is increased from 10° C. to 37° C. then 40° C. In FIG. 15A-B, a peak shift from 122 nm to 459 nm with 1.6-fold greater frequency observed when comparing PMPD2-L10Flag-APPIP at 10° C. versus 37° C. Then from 37° C. to 40° C., in FIG. 15B-C, a further shift is observed with most particle sizes for PMPD2-L10Flag-APPIP at its pI near 531 nm with one major species. This result is like that of the unhindered L10Flag in FIG. 13A-C. The latter confirms the effect and utility of pI-BPS on ELP-based fusion molecules with highly charged ends, to both purify a practical quantity and study the transitioning impedance in an application site at a neutral pH.

### Chronic Wound Mouse Model Results

The in vivo application of this product has been assessed both within fibrin hydrogels as well as a direct application of the dry mixed powder that is subsequently capped with a hydrogel. Both application styles displayed wound closure in 14 days as noted below.

FIG. 16A shows representative images of 2 mice used as a control that were NE pretreated but did not receive a therapeutic treatment. While FIG. 16B displays mice that were NE pretreated and had continuous treatment with NE, but at the start of therapeutic treatments received um-MSC conditioned media and continued therapeutic treatment throughout the experiment. FIG. 16C displays mice that were pretreated and continuously treated with NE, however this mice group received a full therapeutic treatment of both um-MSC conditioned media and the dual inhibitor, PMPD2-L10Flag-APPIP. FIG. 16D displays an image of a mouse that was treated with MSC-CM and dual inhibitor with the wound packed with the therapeutic powder containing tethering peptide.

### Wound Epidermal Thickness and Collagen Composition

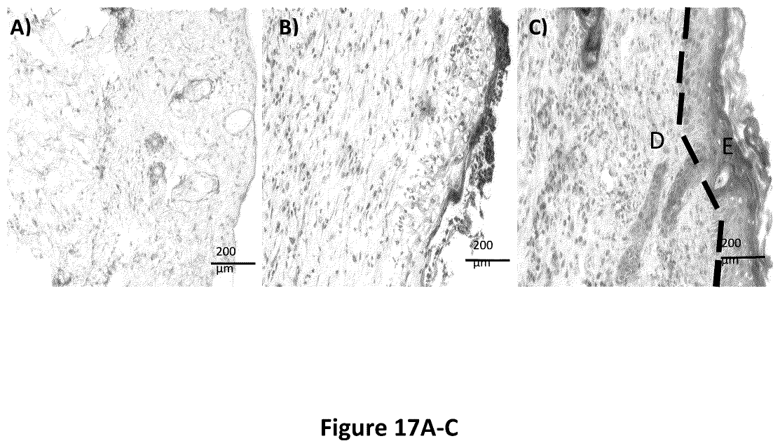

Tissue sections were stained with a Masson's trichrome stain to evaluate the degree of collagen composition and epidermal thickness of the center of each wound. FIG. 17A-C are representative images of mice wound tissue sections that underwent trichrome staining and pictures captured at 40× magnification. A qualitative analysis of these images show, in FIG. 17A-C, stained blue collagen strands between cell nuclei appearing light blue to gray in color with the nuclei of cells staining a dark blue to purple in color. FIG. 28 A-B are images of the untreated control mouse wound and a mouse wound that was treated with just MSC-CM, respectively. When comparing the images in FIG. 17A-B to FIG. 17C it is apparent that normal tissue anatomy with visible layers of the skin are apparent in image C where a full therapeutic treatment of MSC-CM and PMPD2-L10Flag-APPIP was delivered, while in A and B there is a less defined skin anatomy.

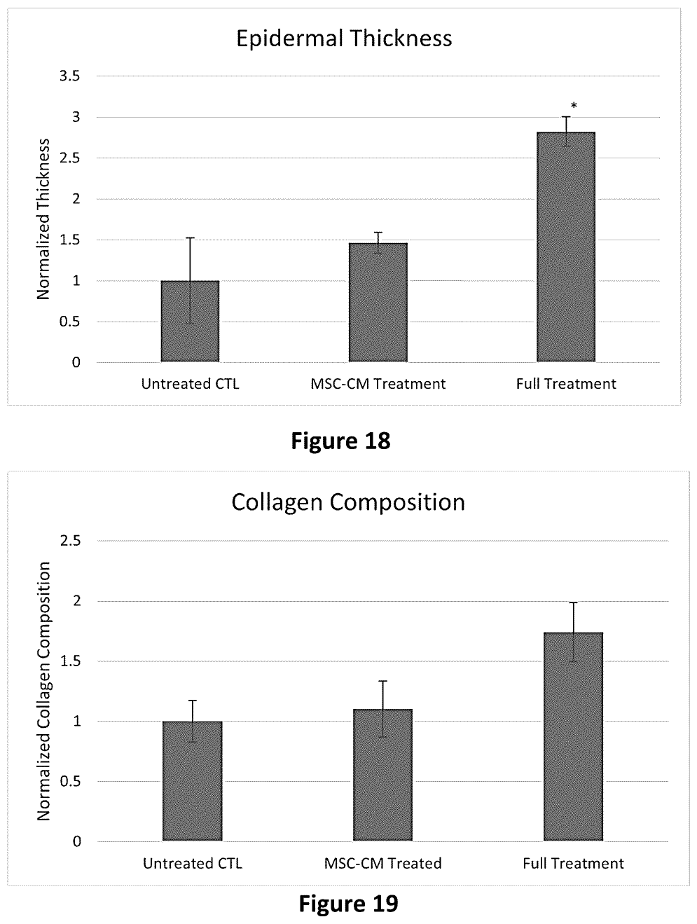

ImageJ was utilized on the 40× microscopy images of trichrome staining of each tissue section from each mouse group, an image from the center of the wound was taken at 40× magnification, the scale was set in ImageJ to align with that of the scale of the 40× images. The epidermis from each image was measured for thickness three separate times across the epidermis of the tissue section to analyze epidermal thickness. The results from the epidermal thickness analysis can be seen in FIG. 18. A 2.8-fold greater epidermal thickness was observed in the full treatment group over that of the untreated control mice. Furthermore, a 1.4-fold greater epidermal thickness was observed in the full treatment group over that of the mice that were treated with just MSC-CM. This result suggests that mice that received the full treatment, MSC-CM and PMPD2-L10Flag-APPIP, healed to a greater degree than the untreated control mice and mice simply treated with MSC-CM.

Additionally, an ImageJ analysis of collagen composition of the trichrome stains further confirms the more developed healing in the full treatment group. FIG. 19 displays the results of average collagen composition of all mice from each group (untreated, MSC-CM treated, and MSC-CM plus the dual inhibitor) and their collagen composition of the wound 14 days following therapeutic treatment. In FIG. 19 it is observed that a 1.7-fold increase in collagen composition, was achieved over the untreated control, observed by the full treatment group. However, the mice group that only received MSC-CM displayed collagen composition in amounts like that of the untreated control mice. Development of more collagen in the mice group that were treated with both MSC-CM and the dual inhibitor further suggests that the untreated control mice and mice just treated with MSC-CM are farther behind in healing than that of the full treatment group.

### Statistical Analysis of Mouse Model Results

A single-factor Anova was ran between groups (Untreated, MSC-CM treated, MSC-CM and PMPD2-L10Flag-APPIP treated) for the ImageJ analysis of collagen composition. The difference of collagen composition between the Untreated controls and the mice treated with MSC-CM were statistically insignificant (P≥0.05, F_{crit}≥F). This statistical result further confirms that there is no difference between mice in this experiment that go untreated and mice that just receive the treatment of MSC-CM and the null hypothesis cannot be rejected. In contrast, statistical significance was found when comparing mice that were treated with both MSC-CM and the dual inhibitor to mice that were untreated or treated with just MSC-CM. A single factor Anova was ran and P-values≤0.05 were obtained with F-critical values less than the F-values. Meeting these two criteria alludes to significant differences between the full treatment group, and the mice that were untreated or treated MSC-CM.

Both the dry MSC conditioned product, the multifunctional inhibitor fusion protein, and the tethering protein can easily be mixed as a dry powder in correct amount that can be verified by in vitro screening of resulting proliferation caused by the final product in the presence of clinically relevant concentrations of proteases. This screening method allows for quality control as the quality of a stem cell conditioned media is reliant on the tight requirements needed in vitro for maximum cellular production of growth factor products by the mesenchymal stem cells.

### Materials and Methods

### Culturing of Mesenchymal Stem Cells

Human umbilical cord derived mesenchymal stem cells (um-MSCs) were obtained from ATCC and plated in a 100 mm dish. The culture was expanded to confluency in 10 mL of basal stem cell culture media, MesenPRO RS (Gibco™), and supplemented with 2% stem-cell qualified FBS. The cells were maintained at 37° C. and 5% CO₂. The MSCs were passed when 80% confluency was reached, utilizing 0.25 trypsin-EDTA, and sub-cultured in 100 mm dishes. It was observed that um-MSC subcultures would expand at a steady rate out to 5 to 6 passages (changing media every 2 days, reaching confluency and passing every 5 to 7 days). Upon media collection and media replacement, 2 mL of media from the last 48-hour incubation was reserved in the dish, and 8 mL of new media was added. Around passage 5, proliferation would slow down and the MSCs would stay at around a 70-80% confluency, without cell death if maintained by the described media replacement every 48-hours.

### Harvesting Um-MSC Conditioned Media

Throughout the expansion process of these um-MSC's, during media replacement (every 48 hours) the media was collected from a MSC culture that was near 70 to 80% confluency and stored to be screened for proliferative effects in vitro. However, it was observed that when the um-MSCs, themselves, slowed down in proliferation they still maintained healthy cellular morphology and attachment. So, a sub-culture of passage 5 um-MSC's were maintained in this state where the MSCs were live and healthy, but not dividing at a steady rate wouldn't lead to another sub-culture. Every 48 hours the media was replaced, and the old media collected and stored at −20° C. In some cases, these slow growing cultures could be maintained for weeks to well over a month while collecting a potent conditioned media, without death of the MSCs.

### Processing the Um-MSC Conditioned Media

Before use of the MSC conditioned media, all samples to be screened were thawed and filtered with a 0.22 μm filter to result in an acellular product, just containing free proteins and soluble small molecules that will remain after filtration. In some cases, the conditioned media was applied in vitro as a liquid to test for its ability to cause proliferation, while in other cases it was freeze dried through lyophilization and applied as a dry product. Additionally, in some experiments, filter paper was soaked in conditioned media, frozen, and then lyophilized to embed the conditioning products within a solid medium.

### Verification of Um-MSCs

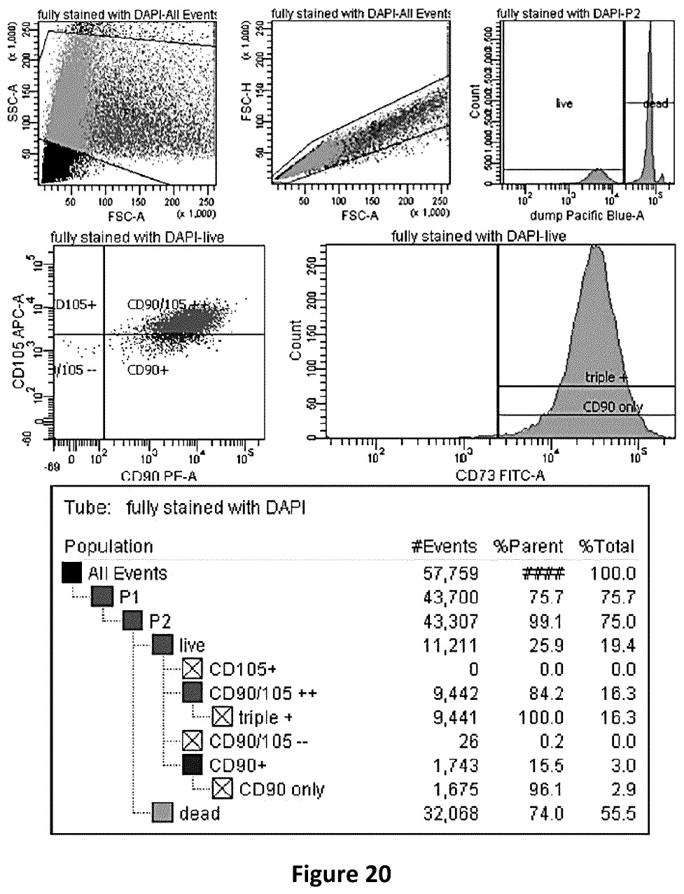

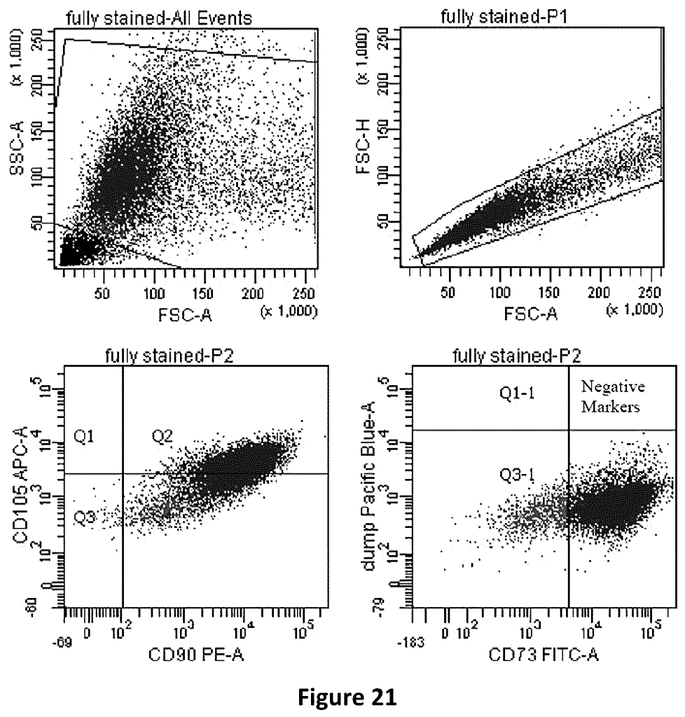

Flow cytometry was employed to verify the mesenchymal stem cells using criteria of cell surface markers to characterize human derived MSCs. MSCs are characterized as positive for CD73, CD90, and CD105 cell surface markers²⁸. Furthermore, MSC characterization guidelines require them to be negative for the cell surface markers CD14, CD34, and CD45²⁸. Conjugated antibodies for CD73, CD90, and CD105 were obtained and utilized with florescent flow cytometry channels for excitation and emission of FITC, thy-1 phycoerythrin, and allophycocyanin respectively. While the negative markers were identified with human Alexa Flour 405 conjugated human antibodies, the same channel as DAPI. DAPI was used in a fully stained sample of MSCs to evaluate viability. A fully stained sample without DAPI added was evaluated for negative marker expression. All negative and positive antibodies were added to a dilute sample of Um-MSCs and analyzed for positive and negative marker expression. To acquire negative marker expression a comparison must be made between a fully-stained sample with DAPI, and a fully stained without the addition of DAPI. FIG. 20 displays the progression of analysis and identification of um-MSC's through the staining of the positive markers CD73, CD90, and CD105 with differing conjugates, as well as DAPI to determine viability of the sample. As it can be seen from the data reported in FIG. 20, cells containing the CD73 positive marker have been identified and exist on the surface of 100% of the live, parent population that expresses both CD90 and 105, with 84% of all live cells detected are positive for all three markers. FIG. 21 displays the flow cytometry results of a fully stained sample without DAPI, but still acquiring measurements utilizing the DAPI channel, for the isolation of cells that may present negative markers disqualifying them as MSCs. In FIG. 21 it is observed that there is no negative marker expression. These results validate the use of these cells as MSCs for conditioning of media. Furthermore, this sample contained a 74% viability post isolation and staining for flow cytometry.

### Screening of Um-MSC Conditioned Media for Inducing Cell Proliferation

C2C12, a mouse muscle myogenic cell line from ATCC was obtained. C2C12 cells have shown quick responsiveness to growth factor induced proliferation and growth as previously used by this laboratory⁴⁴. C2C12 cells were removed from liquid nitrogen storage and seeded at a density of 10⁶ cells per 100 mm dish. The cells were maintained in DMEM 10% FBS supplemented with 1% antibiotic-antimycotic (AA) at 37° C. and 5% CO₂. The C2C12 cells expanded to 80% confluency were then passed to 24-well plates, at a seeding density of 5×10⁴ cells per well, for treatment with MSC conditioned media product.

### Proliferation Experiments Utilizing C2C12 Cells in a Serum-Free Environment

C2C12 cells were plated at a seeding density of 5×10⁴ cells per well and incubated at standard culturing conditions (37° C. and 5% CO₂) over night for attachment. After attachment, the cells in each well were washed three time in 1×PBS, and after the last wash, serum-free DMEM 1% AA was added to each control (n=12) and experimental well (n=12). Then, 0.2 mL of serum-free MesenPRO RS was added to each control well for a final volume of 0.4 mL. MSC conditioned media (200 μl) was then added to each experimental well (n=12), for each conditioned media sample) on the 24-well plate for a total and final volume of 0.4 mL in each well. The assay plate with control wells and treatment wells was then incubated under standard cell culture conditions for 72 hours.

Following the incubation period, the cells were washed three times in 1×PBS and after the last wash 200 μl of deionized water was added to each assay well. Then, a Hoechst assay was employed by utilizing three freeze-thaw procedures to lyse the cells. A 100 μl sample of the lysate was taken from each well and transferred to a corresponding well on a 96-well, round bottom, assay plate. Hoechst 33342, at a working concentration of 0.002 mg/mL was added 1:1 with each lysate sample, with a final volume of 200 μl in each well to be analyzed on the 96-well plate. The assay was then read, Ex/Em 360/460, after a 30 second shaking procedure on a BioTek Synergy HT microplate reader. The resulting data was blanked and analyzed by comparing the degree of florescence, which corresponds to the number of cells in each well, compared to that of the control wells. The average florescent results of the conditioned media treatment wells were divided by the average florescence of the control wells, and multiplied by 100%, to normalize the results to that of the control, and display % growth.

Proliferation Experiments Utilizing C2C12 Cells Treated with MSC-CM and NE

The proliferation experiment using um-MSC conditioned media to induce proliferation, was employed again, but with levels of protease added to the treatment to visualize the affect proteases have on the conditioned media protein products that will fall victim to the inherent threat of proteolytic degradation seen in hard-to-heal wounds. After plating C2C12 cells in a 24-well plate and allowing for overnight attachment in standard cell culture conditions, all wells were washed three times in 1×PBS. Immediately after aspirating the last washing procedure, serum free DMEM with 1% AA was added to each control and experimental well. Utilizing the same conditioned media sample, 0.2 mL of processed conditioned media was added to each treatment well for a total final volume of 0.4 mL. While the control wells (n=6) received 0.2 mL of serum-free MesenPRO RS media. One group of conditioned media treated wells were left unexposed to protease, while other well groups (n=6) received various concentrations of elastase with the largest concentration of elastase being 200 mU/mL, a clinically relevant level of protease¹², to see the true degrading affect that protease levels have on the potential use of conditioned media products in chronic or hard-to-heal wounds. The elastase concentrations tested were 0.02, 0.2, 2, 20, and 200 mU/mL.

After treatment, the 24-well plate was incubated under standard cell culture conditions for 72-hours and then a Hoechst assay was utilized to analyze the proliferative effects of the conditioned media in the presence of protease. The Hoechst assay was performed and analyzed in accordance with the protocol described previously.

### Statistical Analysis for the Proliferation Assays

From all proliferation experiments standard deviation from the average fluorescence from Hoechst assays for each sample was calculated, and % standard deviation was applied to each graphical representation of the data. Additionally, a single factor Anova (p≤0.05) was performed from the data for each experimental MSC-CM treatment group and compared to the control (FIGS. 7A & C). For comparisons between MSC conditioned media groups to their respective untreated control the P-value was p≤0.05 with F≥F-crit for all MSC conditioned media treatments. For FIG. 7B, the single factor Anova was performed between the data from the differing concentrations of NE to the wells that received 0 mU/mL NE, where p≤0.05, F≥F-crit.

### Construction of Recombinant ELP Fusion Proteins

Plasmids containing the genes for L10-Flag, PMP-D2, APP-IP, and PlGF2 were obtained from GenScript®, optimized for corresponding cutting sites for the restriction enzymes PflMI and BglI to exclude cuts within the genes of interest, and includes a gene for resistance to carbenicillin for effective colony isolation. Restriction enzymes (PfLM1 & BglI) and ligase for the recombinant process were obtained by New England Biolabs® Inc. L10FLAG, is denoted by the sequence[(VPGVG)₂(VPGLG)(VPGVG)₂]₁₀ DYKDDDDK (SEQ ID NO: 2). PMP-D2 is denoted by the amino acid sequence EEKCTPGQVKQQDCNTCTCTPTGVWGCTLMGCQPA (SEQ ID NO: 3). APP-IP is denoted by the amino sequence ISYGNDALMP (SEQ ID NO: 4). PlGF2 is denoted by the amino acid sequence RRPKGRGKRREKQRPTDCHL (SEQ ID NO: 5).

The ELP fusion proteins used in this therapeutic mixture were constructed using cloning and recombinant technology in microorganisms. As described in Example 1, pUC57 and pUC19 plasmids containing PMP-D2, APP-IP and L10-Flag were cloned in Top10 and purified to a desirable working concentration for recombinant processes. The plasmid containing L10-Flag was linearized using the restriction enzyme, PflMI. PMP-D2's nucleic acid sequence was cut out of the pUC57 plasmid utilizing both restriction enzymes, PflMI and BglI, and purified to obtain a PMP-D2 fragment. The PMP-D2 fragment and the linearized pUC19 L10-Flag plasmid were then spliced together utilizing recursive directional ligation to obtain the circular plasmid, PMPD2-L10Flag pUC19. After cloning an isolated bacterial colony containing the new plasmid, PMPD2-L10Flag pUC19, the nucleic acid sequence for PMPD2-L10Flag was fragmented out of the pUC19 plasmid. APP-IP in a pUC57 plasmid was linearized and a recursive directional ligation was performed to yield PMPD2-L10Flag-APPIP in a pUC57 plasmid. The gene of interest was then double cut using PflMI and BglI, then spliced into a linearized pET25b plasmid for expression in BLR(DE3) e coli.

The nucleic acid sequence for L10Flag-PlGF2 was made similarly to that of the inhibitor. It was designed so that the ELP region is free for tethering to other proteins. A pUC57 plasmid containing the gene encoding for the PlGF2 protein was linearized. Simultaneously, L10-Flag was fragmented out of a pUC19 plasmid. A recursive directional ligation was performed between L10-Flag fragment and the linearized PlGF2 pUC57. The resulting circular pUC57 plasmid contained the gene encoding for L10Flag-PlGF2. L10Flag-PlGF2 nucleic acid sequence was fragmented from the pUC57 plasmid, ligated into a circular pET25b plasmid, and transformed into BLR(DE3) e coli. All constructed ELP fusion proteins are verified via Sanger Sequencing.

### Expression and Purification of ELP Fusion Proteins

After transformation into BLR(DE3) e coli, a single colony for each desired fusion protein was used to inoculate a separate 75 mL of terrific broth (TB) supplemented with carbenicillin. After 12 hours incubating at 37° C. with agitation, the 75 mL for each protein to be expressed and purified was transferred into a liter of TB supplemented with carbenicillin. A 24-hour incubation at 37° C. with agitation was performed on each culture. After a 24-hour incubation, the cultures were centrifuged to separate the bacteria containing the expressed proteins from the TB. The bacterial pellets were each resuspended in 160 mL of cold 1×PBS and lysed utilizing three sonication and mixing steps. Once lysing is complete the protein and bacterial lysate samples were centrifuged at 4° C., twice, to separate the bacterial debris from the solubilized fusion proteins in the supernatants. Inverse Transition Cycling (ITC) is utilized to separate the ELP fusion proteins from debris. ITC begins by the addition of NaCl to each sample to a final 4 molar concentration. It is important to note, a new procedure was implemented into a standard ITC protocol where before a hot centrifugation, the pH of each sample is adjusted to match the pI of each protein, while before a cold spin the pH is adjusted back to a neutral pH or to a pH extreme depending on protein net protein charge. This process is coined pI-Based Phase Separation (pI-BPS), and necessary to obtain useable protein yields when purifying dual fusions, like PMPD2-L10Flag-APPIP. Each sample was placed in a 45° C. hot bath for 15 to 30 minutes (depending on how quickly each sample transitions). After hot centrifugation, the transitioned protein pellets were separated from the supernatants, and resuspended in 100 mL of cold 1×PBS. Once evenly resuspended and the pH adjustment is made, the samples undergo a cold centrifugation at 4° C. After the cold centrifugation, the supernatants containing the proteins are collected and separated from the pellets containing debris. The hot centrifugation and cold centrifugation alternation and collection with the appropriate pH adjustments are performed repeatedly until the final protein product is cleared of debris. Once resolved, the final protein pellets are resuspended in 50 mL of deionized H₂O and dialysis is performed for 24 hours. Once dialysis is complete, the protein solutions were then frozen at −80° C. and then lyophilized until freeze dried completely.

Verification of pI-Based Phase Separation for the Purification and Application of ELP Fusion Proteins

Protein charge is based on the charge of each residue that comprises the overall amino acid sequence for a particular protein. Isoelectric point (pI) is a solution pH where a protein exhibits a net zero charge. PI-BPS was verified useful through experimental observation of protein yields resulting from inverse transition cycling, quantification of transitioning at a neutral pH and at a protein's pI evaluated by absorbance over temperature changes, and dynamic light scattering. Table 3 displays ELP fusion proteins, their sequences, and isoelectric point tested in the absorbance-based transitioning experiments, while not all were evaluated by dynamic light scattering.

TABLE 3
Complete fusion protein sequences and their
isoelectric points for each
protein studied using pI-BPS

| | | Isoelectric |
|---|---|---|
| Protein | Sequence | Point (pI) |
| L10Flag | ((VPGVG)2(VPGLG) | 3.74 |
| SEQ ID NO: 2 | (VPGVG)2)10-DYKD | |
| | DDDK | |
| PMPD2- | EEKCTPGQVKQQDCNT | 4.10 |
| L10Flag- | CTCTPTGVWGCTLMGC | |
| APPIP | QPA-((VPGVG)2 | |
| SEQ ID NO: 6 | (VPGLG)(VPGVG)2)10- | |
| | DYKDDDDK-ISYGNDALM | |
| | P | |
| L10Flag- | ((VPGVG)2(VPGLG) | 9.35 |
| P1GF2 | (VPGVG)2)10-DYKDDD | |
| SEQ ID NO: 7 | DK-RRPKGRGKRR | |
| | REKQRPTDCHL | |

### Quantifying ELP Fusion Protein Transitioning Utilizing Absorbance Measurements

Various ELP fusion proteins were reconstituted at a neutral pH and at a specific protein's isoelectric point, where the net charge of the entire protein molecule is zero. Utilizing the known amino acid sequences for each ELP fusion protein and an online (isoelectric.org) isoelectric point calculator to calculate the pH where each protein of interest has a zero net charge. The isoelectric point calculator is based upon the use of Henderson-Hasselbach equation, and dissociation constants of positive and negative residues to approximate a protein's charge at a given pH. PMPD2-L10Flag-APPIP and other various proteins were solubilized at its calculated pI and at 7.4 pH. Then in a 96-well plate, the samples were arranged in triplicate. Due to the experimental design and nature of transitioning experiments, correlated blanks of each buffer pH were included. The plate with samples were placed in a BioTek Synergy HT microplate reader, utilizing the software, Gen5, for a kinetic assay, varying temperature overtime while recording OD360. The temperature start point was 30° C. and the 96-well plate was preheated to the start temperature before the first reading. The following temperature and read points were at 35, 40, and 45° C. Before each read, there was a delay to account for machine warm up and time for sample transitioning at a particular temperature.

### Dynamic Light Scattering Experiments

Dynamic Light Scattering (DLS) was performed on samples of both L10Flag and PMPD2-L10Flag-APPIP to better understand the phase separation behavior of these ELP fusion proteins at the protein's isoelectric points and in a neutral pH. A Malvern Zetasizer ZEN 1600, by Malvern Panalytical Ltd., was used to perform all DLS experiments. The DLS protocol was designed to measure size distribution of assembling ELP fusion proteins over a series of temperatures. The protocol begins with a temperature set point of 10° C., then increasing the temperature in 5° C. increments with the last DLS measurement at 45° C. After the sample reaches each temperature set point, size distribution and correlogram data was measured and recorded. This experiment is performed on both L10Flag at a neutral pH and PMPD2-L10Flag-APPIP at a neutral pH and at its pI. The resulting DLS data is analyzed in Excel to plot size distribution and associated correlogram data.

### Chronic Wound Diabetic Mouse Model

A combination of um-MSC conditioned media and the dual protease inhibitor were tested for wound healing capabilities, in vivo, in a diabetic chronic wound mouse model. The necessity for this combination has been shown valuable in vitro, but it is imperative that this combination is tested in a real wound environment. The animal model protocol run here, followed regulatory, timeframe, and animal safety guidelines set out by the University of South Florida's Institutional Animal Care and Use Committee (IACUC) before, during, and after the animal experiment was employed. Nine diabetic mice were obtained from The Jackson Laboratory. All mice ordered and received from The Jackson Laboratory were female and of the strain 000697, a B6.BKS (D)-Lepr db/that were 6-8 weeks old. Upon arrival the mice were left to acclimate for 7 days before a wounding procedure was employed. Fourteen days after the initial therapeutic treatments, the mice were euthanized, and tissue samples were collected from the wound of each mouse to evaluate whether reepithelization and matrix deposition occurred when treated with the combination of PMPD2-L10Flag-APPIP and um-MSC conditioned media.

### Surgical Wounding Procedure

After mice arrival and a 7-day acclimation period, the mice underwent the following wounding procedure. One mouse at a time was anesthetized utilizing isoflurane and shaved on the middle of the back of the mouse. Once shaved, if there was a large density of hair on the mouse, Nair was utilized to make the back of the mouse bald in the area of incision. It is important to get a clean shave to ensure a durable application of the bandages at the end of the wounding procedure. Once cleanly shaved and sterilized with an alcohol pad, a 1 cm×1 cm square, scored with a permanent marker was applied. The incision was made utilizing 11.5 cm straight blade Metzenbaum scissors to cut along the scored 1 cm×1 cm square on the skin of the mouse. The incision was started at a corner of the square by pulling up one corner with Graefe forceps and making the initial cut with the skin tuff pulled off the back of the mouse, and then cutting along the rest of the scoring lines. Once the 1 cm×1 cm incision is made the resulting square piece of skin was removed from the back of the mouse.

### NE Pretreatment for Modeling and Inducing a Chronic Wound Environment

Nine mice were pretreated with NE for 7 days to model a chronic wound environment before therapeutic treatments. Fractions of fibrinogen and thrombin were prepared to then mix to become a fibrin gel for the delivery of both NE and the therapeutic treatments. The fibrinogen fraction was prepared by making a stock solution at a concentration of 62.5 mg/mL. The working fibrinogen solution is achieved by diluting the stock 1:10 in 1×PBS. While the thrombin working solution was made from the mixing of 50 μl of 100 U/mL solution of thrombin, 5 μl of 1 M CaCl₂), and 345 μl of deionized H₂O. During wounding the thrombin fraction was placed on ice and the fibrinogen fraction was placed in a 37° C. water bath. When ready to apply a fibrin gel to the wound on the back of the mouse, 20 μl of the thrombin fraction is combine with 80 μl of the fibrinogen fraction and mixed. NE was added to the fibrin gel to a final NE concentration in the 100 μl gel of 250 mU/mL (a clinically relevant concentration in chronic wounds) and then applied to the wound and let to solidify for 2 minutes. The mouse was then bandaged, removed from the isoflurane, and observed until fully awake. This NE pretreatment for 9 mice was done twice in the 7-day pretreatment period and then therapeutic treatments were employed.

Treating the Mice Wounds with Fibrin Gels Containing the Dual Protease Inhibitor and Um-MSC Conditioned Media

Fibrin gel preparation here follows the procedure outlined in section NE Pretreatment for Modeling and Inducing a Chronic Wound Environment, however PMPD2-L10Flag-APPIP is added to the fibrinogen fraction for 3 mice receiving a full treatment of both inhibitor and um-MSC conditioned media, with a final concentration of 1 μg/μl. Um-MSC conditioned media was dialyzed, frozen, and lyophilized. The resulting powder was added into the fibrinogen fraction at a concentration of 0.5 μg/μl, in accordance with the conditioned media volume that showed results in the in vitro proliferation studies. Before treatment, the thrombin fraction was placed on ice, while the fibrinogen fraction containing um-MSC conditioned powder, and the dual protease inhibitor was placed in a 37° C. bath. The fibrin gel was made by adding 20 μl of the thrombin fraction to 80 μl of the fibrinogen/inhibitor/conditioned media fraction. Then it was lightly mixed and NE was added to the gel for a final concentration of 250 mU/mL. Next, the gel was added to the wound and let to solidify for 2 minutes. After the gel was solidified, bandages were applied, and the mouse was observed until awakening from the anesthesia. Table 4 summarizes the treatment groups.

TABLE 4
Treatment groups and controls for the chronic wound mouse model

| Treatment | |
|---|---|
| Groups | Format |
| Negative Control | 7-Day NE Pretreatment + Continuous NE Treatment |
| MSC-CM Treated | 7-Day NE Pretreatment + Continuous NE Treatment |
| | with Um-MSC Conditioned Powder |
| Full Treatment | 7-Day NE Pretreatment + Continuous NE Treatment |
| | with Um-MSC Conditioned Powder and the Dual |
| | Protease Inhibitor |

### Tissue Collection and Histology (Trichome Stains for Wound Anatomy, Collagen Quantification, Epidermal Thickness Quantification)

Fourteen days from the application of therapy, 21 days from NE pretreatment, the mice were euthanized, and wound tissue was collected. The mice were euthanized using a CO₂ flow rate of 10%-30% of cage volume per minute and a secondary means of euthanizing was employed via cervical dislocation. The wound tissues were collected after each mouse was euthanized, by cutting around the wound, leaving a halo of healthy tissue, with Metzenbaum scissors and pulling the entire circular wound off the back of each mouse. The wound was cut in half down the diameter of the tissue sample. Half of the wound was placed in a specimen mold with OCT compound with the wound edge placed down for tracking orientation for tissue sectioning. Immediately, the tissue sample in the cryomold was placed on dry ice for flash freezing, and then stored in −80° C. until sectioning. The sectioning was performed by Moffit Cancer Center's histology core. Tissue samples were sectioned to obtain a full tissue section of the cross section of the wound. Trichrome stains were employed and analyzed in microscopy to evaluate the degree of healing through identifiable features such as re-epithelialization and collagen matrix formation in the wound samples.

### CONCLUSION

The inventors have developed a novel therapeutic agent for the treatment of chronic and hard to heal wounds comprised of three main components: a multifunctional protease inhibitor, a conditioned media, and a tethering peptide. The multifunctional protease inhibitor is a single protein comprised of different protease inhibiting peptides each separated by an ELP. This allows for a single protein to bind to different proteases thus inhibiting them and promoting wound closure. The conditioned media provides much needed growth factors to aid in wound closure. The tethering peptide acts as a tether to allow the multifunctional protease inhibitor to stay in contact with the wound bed for sustained release. The therapeutic agent allows for a multipronged approach in treating chronic and hard to heal wounds that increases wound healing time.

### Example 3—Treatment of Chronic Wound (Prophetic)

A 56-year-old male presents with a wound that has been open for over 3 months. A therapeutically effective amount of a composition comprising a multifunctional protease inhibitor (PMP-D2-L10f-APP-IP); conditioned media from stem cells; and a tethering peptide (L10f-PlFG2) is provided as a dry powder mixture. The therapeutic agent is reconstituted into a fibrin gel and applied to the wound daily for 2 weeks. At recheck, the wound is markedly improved with re-epithelization apparent. The composition continues to be applied until complete wound closure.

### Example 4—Treatment of Chronic Wound with Infection (Prophetic)

A 64-year-old male presents with a wound that has failed to close for a month. The wound shows signs of infection. A therapeutically effective amount of a composition comprising a multifunctional protease inhibitor (PMP-D2-L10f-APP-IP); conditioned media from stem cells; an antimicrobial fusion peptide; and a tethering peptide (L10f-PlFG2) is provided as a dry powder mixture. The wound is packed with the powder mixture daily and monitored. After 2 weeks, signs of infection are diminished and re-epithelization is apparent.

### Example 5—Preventing Rejection of Implant (Prophetic)

A 70-year-old woman presents with severe hip pain and is unable to climb stairs or tie her shoes. Testing is conducted and a hip replacement is recommended. The dry composition comprised of a multifunctional protease inhibitor (PMP-D2-L10f-APP-IP); conditioned media from stem cells; and a tethering peptide (L10f-PlFG2) is incorporated into the replacement hip during manufacturing. The hip is replaced and at recheck, the woman shows no signs of implant rejection.

The sequence listing entitled “Protein Based Advanced Wound Healing System” in XML format, created on Aug. 18, 2023 and being 8,000 bytes in size, is hereby incorporated by reference into this disclosure.

### REFERENCES

1. van Hasselt J G C, Iyengar R. Systems pharmacology: defining the interactions of drug combinations. Annu Rev Pharmacol Toxicol. 2019; 59:21-40.
2. Schultz G S, Wysocki A. Interactions between extracellular matrix and growth factors in wound healing. Wound Repair Regen. 2009; 17(2): 153-162.
3. Afonso A C, Oliveira D, Saavedra M J, Borges A, Simões M. Biofilms in diabetic foot ulcers: impact, risk factors and control strategies. Int J Mol Sci. 2021; 22 (15): 8278.
4. González-Pérez F, Ibáñez-Fonseca A, Alonso M, Rodríguez-Cabello J C. Combining tunable proteolytic sequences and a VEGF-mimetic peptide for the spatiotemporal control of angiogenesis within elastin-like Recombinamer scaffolds. Acta Biomater. 2021; 130:149-160.
5. Baker E A, Leaper D J. Proteinases, their inhibitors, and cytokine profiles in acute wound fluid. Wound Repair Regen. 2000; 8(5): 392-398.
6. McCarty S M, Percival S L. Proteases and delayed wound healing. Adv Wound Care (New Rochelle). 2013; 2(8): 438-447.
7. Okonkwo U A, DiPietro L A. Diabetes and wound angiogenesis. Int J Mol Sci. 2017; 18 (7): 1419.
8. Han G, Ceilley R. Chronic wound healing: a review of current management and treatments. Adv Ther. 2017; 34(3): 599-610.
9. Sutcliffe J E S, Thrasivoulou C, Serena T E, et al. Changes in the extracellular matrix surrounding human chronic wounds revealed by 2-photon imaging: second harmonic imaging of chronic wound extracellular matrix. Int Wound J. 2017; 14 (6): 1225-1236.
10. Armstrong D G, Jude E B. The role of matrix metalloproteinases in wound healing. J Am Podiatr Med Assoc. 2002; 92(1): 12-18.
11. Bergant Suhodolc an A, Luzar B, Kecelj L N. Matrix metalloproteinase (MMP)-1 and MMP-2, but not COX-2 serve as additional predictors for chronic venous ulcer healing. Wound Repair Regen. 2021; 29(5): 725-731.
12. Boeringer T, Gould L J, Koria P. Protease-resistant growth factor formulations for the healing of chronic wounds. Adv Wound Care (2162-1918). 2020; 9(11): 612.
13. Ferreira A V, Perelshtein I, Perkas N, Gedanken A, Cunha J, Cavaco-Paulo A. Detection of human neutrophil elastase (HNE) on wound dressings as marker of inflammation. Appl Microbiol Biotechnol. 2017; 101(4): 1443-1454.
14. Serena T E, Cullen B M, Bayliff S W, et al. Defining a new diagnostic assessment parameter for wound care: elevated protease activity, an indicator of nonhealing, for targeted protease-modulating treatment. Wound Repair Regen. 2016; 24(3): 589-595.
15. Heredero M, Garrigues S, Gandía M, Marcos J, Manzanares P. Rational design and biotechnological production of novel AfpBPAF26 chimeric antifungal proteins. Microorganisms. 2018; 6(4): 106.
16. McCarthy B, Yuan Y, Koria P. Elastin-like-polypeptide based fusion proteins for osteogenic factor delivery in bone healing. Biotechnol Prog. 2016; 32(4): 1029-1037.
17. Bhorkar I, Dhoble A S. Advances in the synthesis and application of self-assembling biomaterials. Prog Biophys Mol Biol. 2021.
18. Urry D W. Physical chemistry of biological free energy transduction as demonstrated by elastic protein-based polymers. J Phys Chem B. 1997; 101(51): 11007-11028.
19. Meyer D E, Chilkoti A. Genetically encoded synthesis of protein-based polymers with precisely specified molecular weight and sequence by recursive directional ligation: examples from the elastin-like polypeptide system. Biomacromolecules. 2002; 3(2): 357-367.
20. Meyer D E, Chilkoti A. Purification of recombinant proteins by fusion with thermally responsive polypeptides. Nat Biotechnol. 1999; 17(11): 1112-1115.
21. Johnson T, Koria P. Expression and purification of neurotrophin-elastin-like peptide fusion proteins for neural regeneration. BioDrugs: Clin Immunother Biopharm Gene Ther. 2016; 30(2): 117-127.
22. Leonard A, Koria P. Growth factor functionalized biomaterial for drug delivery and tissue regeneration. J Bioact Compat Polym. 2017; 32(6): 568-581.
23. Qin J, Luo T, Kiick K L. Self-assembly of stable Nanoscale platelets from designed elastin-like peptide-collagen-like peptide bioconjugates. Biomacromolecules. 2019; 20(4): 1514-1521.
24. Koria P, Yagi H, Kitagawa Y, et al. Self-assembling elastin-like peptides growth factor chimeric nanoparticles for the treatment of chronic wounds. Proc Natl Acad Sci U SA. 2011; 108(3): 1034-1039.
25. Vasconcelos A, Azoia N G, Carvalho A C, Gomes A C, Güebitz G, Cavaco-Paulo A. Tailoring elastase inhibition with synthetic peptides. Eur J Pharmacol. 2011; 666(1): 53-60.
26. Ndinguri M W, Bhowmick M, Tokmina-Roszyk D, Robichaud T K, Fields G B. Peptide-based selective inhibitors of matrix metalloproteinase mediated activities. Molecules. 2012; 17(12):14230.
27. Monfort D A, Koria P. Recombinant elastin-based nanoparticles for targeted gene therapy. Gene Ther. 2017; 24(10):610-620.
28. Smeets R, Ulrich D, Unglaub F, Wöltje M, Pallua N. Effect of oxidized regenerated cellulose/collagen matrix on proteases in wound exudate of patients with chronic venous ulceration. IntWound J. 2008; 5(2):195-203.
29. van Hinsbergh V W, Collen A, Koolwijk P. Role of fibrin matrix in angiogenesis. Ann N Y Acad Sci. 2001; 936:426-437.
30. Lipsky B A, Berendt A R, Deery H G, et al. Diagnosis and treatment of diabetic foot infections. Clin Infect Dis. 2004; 39(7): 885-910.
31. Cole A M, Shi J, Ceccarelli A, Kim Y H, Park A, Ganz T. Inhibition of neutrophil elastase prevents cathelicidin activation and impairs clearance of bacteria from wounds. Blood. 2001; 97(1): 297-304.
32. Hosseinkhani H, Hosseinkhani M, Khademhosseini A, Kobayashi H, Tabata Y. Enhanced angiogenesis through controlled release of basic fibroblast growth factor from peptide amphiphile for tissue regeneration. Biomaterials. 2006; 27(34): 5836-5844.
33. Yu G, Baeder D Y, Regoes R R, Rolff J. Combination effects of antimicrobial peptides. Antimicrob Agents Chemother. 2016; 60(3): 1717-1724.
34. Atefyekta S, Pihl M, Lindsay C, Heilshorn S C, Andersson M. Antibiofilm elastin-like polypeptide coatings: functionality, stability, and selectivity. Acta Biomater. 2019; 83:245-256.
35. Mikaël MM, Priscilla S B, Esra G, et al. Growth Factors Engineered for Super-Affinity to the Extracellular Matrix Enhance Tissue Healing. Science. 2014; 343(6173):885-888.
36. Lindsay, S., Oates, A. & Bourdillon, K. The detrimental impact of extracellular bacterial proteases on wound healing. International wound journal 14, 1237-1247 (2017).
37. Supuran, C. T., Scozzafava, A. & Mastrolorenzo, A. Bacterial proteases: current therapeutic use and future prospects for the development of new antibiotics. Expert Opinion on Therapeutic Patents 11, 221-259 (2001).
38. Demidova-Rice, T. N., Hamblin, M. R. & Herman, I. M. Acute and impaired wound healing: pathophysiology and current methods for drug delivery, part 1: normal and chronic wounds: biology, causes, and approaches to care. Adv Skin Wound Care 25, 304-314 (2012).
39. Barrientos, S., Stojadinovic, O., Golinko, M. S., Brem, H. & Tomic-Canic, M. Growth factors and cytokines in wound healing. Wound Repair Regen 16, 585-601 (2008).
40. Berebichez-Fridman, R. et al. The Holy Grail of Orthopedic Surgery: Mesenchymal Stem Cells-Their Current Uses and Potential Applications. Stem Cells Int 2017, 2638305-2638305 (2017).
41. Chen, W. et al. Angiogenic and osteogenic regeneration in rats via calcium phosphate scaffold and endothelial cell co-culture with human bone marrow mesenchymal stem cells (MSCs), human umbilical cord MSCs, human induced pluripotent stem cell-derived MSCs and human embryonic stem cell-derived MSCs. J Tissue Eng Regen Med 12, 191-203 (2018).
42. Zhang, J. et al. Exosomes released from human induced pluripotent stem cells-derived MSCs facilitate cutaneous wound healing by promoting collagen synthesis and angiogenesis. J Transl Med 13, 49 (2015).
43. Pereira, T. et al. MSCs conditioned media and umbilical cord blood plasma metabolomics and composition. PLoS One 9, e113769-e113769 (2014).
44. Koria, P. Delivery of growth factors for tissue regeneration and wound healing. BioDrugs: clinical immunotherapeutics, biopharmaceuticals and gene therapy, 163 (2012).

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A composition comprising:
conditioned media from stem cells;
at least one multifunctional protease inhibitor; and
at least one tethering peptide.

2. The composition of claim 1, further comprising at least one antimicrobial fusion peptide.

3. The composition of claim 1, wherein the stem cells are mesenchymal stem cells derived from the umbilical cord.

4. The composition of claim 1, wherein the conditioned media comprises at least one growth factor.

5. The composition of claim 1, wherein the at least one multifunctional protease inhibitor is comprised of an elastase inhibitor attached to one end of an elastin-like peptide (ELP) with a matrix metalloproteinase inhibitor (MMPI) attached to an opposing end of the ELP.

6. The composition of claim 5, wherein the elastase inhibitor is PARS intercerebralis major peptide D2 (PMP-D2).

7. The composition of claim 5, wherein the MMPI is β-amyloid precursor protein-derived inhibitory peptide (APP-IP).

8. The composition of claim 5, wherein the ELP of the multifunctional protease inhibitor is L10flag.

9. The composition of claim 1, wherein the tethering peptide comprises an extracellular matrix (ECM) peptide attached to an elastin-like peptide (ELP).

10. The composition of claim 9, wherein the ECM peptide is placental growth factor 2 (PlGF2) and the ELP of the tethering peptide is L10flag.

11. A method of treating a wound in a patient in need thereof comprising:
administering to the patient a therapeutically effective amount of a composition comprising
conditioned media from stem cells wherein the conditioned media comprises at least one growth factor;
at least one multifunctional recombinant protease inhibitor comprised of an elastase inhibitor attached to one end of an elastin-like peptide (ELP) with a matrix metalloproteinase inhibitor (MMPI) attached to an opposing end of the ELP.; and
at least one tethering peptide;
wherein administration of the composition enhances wound healing to treat the wound.

12. The method of claim 11, wherein the composition further comprises at least one antimicrobial fusion peptide.

13. The method of claim 11, wherein the stem cells are umbilical cord derived mesenchymal stem cells.

14. The method of claim 11, wherein the elastase inhibitor is PARS intercerebralis major peptide D2 (PMP-D2), the MMPI is β-amyloid precursor protein-derived inhibitory peptide (APP-IP) and the ELP of the multifunctional protease inhibitor is L10flag.

15. The method of claim 11, wherein the tethering peptide comprises an extracellular matrix (ECM) peptide attached to an elastin-like peptide (ELP).

16. The method of claim 15, wherein the ECM peptide is placental growth factor 2 (PlGF2) and the ELP of the tethering peptide is L10flag.

17. A multifunctional protease inhibitor comprising:
at least two bioactive molecules wherein one of the at least two bioactive molecules is an elastase inhibitor and one of the at least two bioactive molecules is a matrix metalloproteinase inhibitor (MMPI); and
at least one elastin-like peptide (ELP) bound at each end to one of the at least two bioactive molecules.

18. The multifunctional protease inhibitor of claim 17, wherein the elastase inhibitor is PARS intercerebralis major peptide D2 (PMP-D2).

19. The multifunctional protease inhibitor of claim 17, wherein the MMPI is β-amyloid precursor protein-derived inhibitory peptide (APP-IP).

20. The multifunctional protease inhibitor of claim 17, wherein the ELP is L10flag.


SEQUENCE LISTING (7 sequences)

SEQ ID NO: 1 - protein, 5 aa, synthetic construct
VPGXG

SEQ ID NO: 2 - protein, 258 aa, synthetic construct
VPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGDYKDDDDK

SEQ ID NO: 3 - protein, 35 aa, Locusta migratoria
EEKCTPGQVKQQDCNTCTCTPTGVWGCTLMGCQPA

SEQ ID NO: 4 - protein, 10 aa, Homo sapiens
ISYGNDALMP

SEQ ID NO: 5 - protein, 20 aa, Homo sapiens
RRPKGRGKRREKQRPTDCHL

SEQ ID NO: 6 - protein, 303 aa, synthetic construct
EEKCTPGQVKQQDCNTCTCTPTGVWGCTLMGCQPAVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGDYKDDDDKISYGNDALMP

SEQ ID NO: 7 - protein, 278 aa, synthetic construct
VPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGVPGVGVPGVGVPGLGVPGVGVPGVGDYKDDDDKRRPKGRGKRREKQRPTDCHL